SEQUENCE LISTING (26 sequences)

SEQ ID NO: 1 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 449 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 219 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNADDDDDDQSGNSQESVTEQDSKDSTYSLSSTLTLSKADYEKHKVYACEVTHQGLSSPVTKSFNRGEC

SEQ ID NO: 4 - protein, 449 aa, synthetic construct; identical to SEQ ID NO: 2

SEQ ID NO: 5 - protein, 214 aa, synthetic construct; identical to SEQ ID NO: 1

SEQ ID NO: 6 - protein, 684 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGADDDDDDGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - protein, 214 aa, synthetic construct; identical to SEQ ID NO: 1

SEQ ID NO: 8 - protein, 455 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGDDDDDD

SEQ ID NO: 9 - protein, 219 aa, synthetic construct; identical to SEQ ID NO: 3

SEQ ID NO: 10 - protein, 684 aa, synthetic construct; identical to SEQ ID NO: 6

SEQ ID NO: 11 - protein, 219 aa, synthetic construct; identical to SEQ ID NO: 3

SEQ ID NO: 12 - protein, 455 aa, synthetic construct; identical to SEQ ID NO: 8

SEQ ID NO: 13 - protein, 214 aa, synthetic construct; identical to SEQ ID NO: 1

SEQ ID NO: 14 - protein, 690 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGADDDDDDGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGDDDDDD

SEQ ID NO: 15 - protein, 219 aa, synthetic construct; identical to SEQ ID NO: 3

SEQ ID NO: 16 - protein, 690 aa, synthetic construct; identical to SEQ ID NO: 14

SEQ ID NO: 17 - DNA, 32 bp, synthetic construct
  source 1..32: mol_type = other DNA
gatgatgacgacgacgactgagcggccgctcg

SEQ ID NO: 18 - DNA, 40 bp, synthetic construct
  source 1..40: mol_type = other DNA
ctcagtcgtcgtcgtcatcatcacccggagacagggagag

SEQ ID NO: 19 - DNA, 37 bp, synthetic construct
  source 1..37: mol_type = other DNA
gatgatgatgacgacgatcaatcgggtaactcccagg

SEQ ID NO: 20 - DNA, 44 bp, synthetic construct
  source 1..44: mol_type = other DNA
ccgattgatcgtcgtcatcatcatcggcgttatccaccttccac

SEQ ID NO: 21 - DNA, 35 bp, synthetic construct
  source 1..35: mol_type = other DNA
gacgacgatgacgacgatggcgtgcacaccttccc

SEQ ID NO: 22 - DNA, 44 bp, synthetic construct
  source 1..44: mol_type = other DNA
gcacgccatcgtcgtcatcgtcgtcggcgcctgagttccacgac

SEQ ID NO: 23 - DNA, 29 bp, synthetic construct
  source 1..29: mol_type = other DNA
gtcgaacttttcgatcagaaacttctcga

SEQ ID NO: 24 - DNA, 29 bp, synthetic construct
  source 1..29: mol_type = other DNA
tcgagaagtttctgatcgaaaagttcgac

SEQ ID NO: 25 - protein, 297 aa, Homo sapiens
MRIAVICFCLLGITCAIPVKQADSGSSEEKQLYNKYPDAVATWLNPDPSQKQNLLAPQTLPSKSNESHDHMDDMDDEDDDDHVDSQDSIDSNDSDDVDDTDDSHQSDESHHSDESDELVTDFPTDLPATEVFTPWPTVDTYDGRGDSWYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLWDPKSKEEDKHLKFRISHELDSASSEVN

SEQ ID NO: 26 - protein, 316 aa, Homo sapiens
MKTAULLSILGMACAFSMKNLHRRVKIEDSEENGVFKYRPRYYLYKHAYFYPHLKRFPVQGSSDSSEENGDDSSEEEEEEEETSNEGENNEESNEDEDSEAENTTLSATTLGYGEDATPGTGYTGLAAIQLPKKAGDITNKATKEKESDEEEEEEEEGNENEESEAEVDENEQGINGTSTNSTEAENGNGSSGEDNGEEGEEESVTGANAEGTTETGGQGKGTSKTTTSPNGGFEPTTPPQVYRTTSPPFGKTTTVEYEGEYEYTGANDYDNGYEIYESENGEPRGDNYRAYEDEYSYFKGQGYDGYDGQNYYHHQ

(19) United States
(12) Patent Application Publication
(10) Pub. No.: US 2025/0084183 A1
(43) Pub. Date: Mar. 13, 2025

(54) ENGINEERED COMPOSITIONS FOR BONE-TARGETED THERAPY

(71) Applicant: William Marsh Rice University, Houston, TX (US)

(72) Inventors: Han XIAO, Houston, TX (US); Chenfei YU, Houston, TX (US); Zeru TIAN, Houston, TX (US)

(73) Assignee: William Marsh Rice University, Houston, TX (US)

(21) Appl. No.: 18/580,548

(22) PCT Filed: Jul. 20, 2022
(86) PCT No.: PCT/US2022/073940

§ 371 Date: Jan. 18, 2024

Related U.S. Application Data
(60) Provisional application No. 63/223,875, filed on Jul. 20, 2021.

(51) Int. Cl. C07K 16/32; A61K 39/00; A61K 47/68; A61P 35/04; C07K 14/54; C07K 16/28

(52) U.S. Cl. CPC C07K 16/32; A61K 47/68031; A61K 47/6851; A61P 35/04; C07K 14/5412; C07K 16/2896; A61K 2039/505; C07K 2317/73; C07K 2317/92

(57) ABSTRACT

The present disclosure provides methods for treating bone disease, such as bone cancers or bone metastasis of cancers, by administering an engineered bone-targeting compositions, such as an antibody or one or more polypeptides, such as an antibody or polypeptide engineered to comprise bone-homing peptide(s). Further provided herein are bone-targeting compositions, such as an antibody or one or more polypeptides, such as an antibody or polypeptide engineered to comprise bone-homing peptide(s).

### PRIORITY CLAIM

This application claims benefit of priority to U.S. Provisional Application Ser. No. 63/223,875, filed Jul. 20, 2021, the entire contents of which are hereby incorporated by reference.

### INCORPORATION OF SEQUENCE LISTING

This application contains a Sequence Listing XML, which has been submitted electronically and is hereby incorporated by reference in its entirety. Said XML Sequence Listing, created on Jul. 18, 2022, is named RICEP0085WO.xml and is 30,327 bytes in size.

### BACKGROUND

### 1. Field

The disclosure relates generally to the field of molecular biology. More particularly, it concerns methods of site-specific delivery of an antibody or polypeptide.

### 2. Related Art

Antibody-based therapies entered the clinic over 30 years ago and have become the mainstream therapeutic option for patients with malignancies,^{1,2} infectious diseases,^{3,4} and transplant rejection.⁵ Compared with traditional chemotherapy, these biotherapeutics preferentially target cells presenting tumor-associated antigens, resulting in improved treatment outcomes and reduced side effects.^{6,7,8,9} Despite their high affinity for tumor antigens, poor tumor tissue penetration and heterogeneous distribution of therapeutic antibodies in brain and bone have significantly limited their efficacy in treating diseases in these tissues. Failure to deliver efficacious antibody doses throughout the tumor in these tissues leads not only to treatment failure, but also to development of acquired drug resistance.¹⁰ Exposure to subtherapeutic antibody levels has been shown to facilitate tumor cell ability to evade antibody-mediated killing.^{11,12} Furthermore, attempts to ensure effective concentrations of antibodies in the tumor niche usually leads to high concentrations in other tissues, resulting in adverse systemic side effects that may limit or exclude use of the therapeutic. Thus, strategies to improve tumor penetration and distribution of antibodies in a specific tissue following systemic delivery are needed for optimizing the clinical potential of these agents.

Despite a 5-year survival rate greater than 90%, between 20-40% of breast cancer survivors will eventually experience metastases to distant organs, even years after the initial treatment.¹³ Bone is the most frequent tissue for breast cancer metastases.^{14,15} Dosing the bone microenvironment has proved to be difficult due to the relatively low density of vascularization and the presence of physical barriers to penetration. Antibody-based therapies face special distribution difficulties due to the large molecular size of these agents. Thus, therapeutic antibodies that exhibit excellent efficacy for the treatment of primary mammary tumors yield only suboptimal responses in patients with bone metastases. For example, the trastuzumab (Herceptin) antibody that successfully targets human epidermal growth factor receptor 2 (HER2) in primary breast tumors has also been evaluated as a treatment option for patients with metastatic breast cancer. Although some breast cancer patients benefit from these treatments, a large number of breast cancer patients with bone metastasis experience further tumor progression within one year, and few patients achieve prolonged remission. 16 Thus, the efficacy of therapeutic antibodies appears to be particularly limited in the case of bone metastases.

### SUMMARY

In certain embodiments, the present disclosure provides methods for a bone-targeting polypeptide or protein (e.g., an antibody) engineered to comprise at least one bone-homing peptide which selectively binds to bone hydroxyapatite (HA). In one embodiment, the present disclosure provides methods for a bone-targeting antibody engineered to comprise at least one bone-homing peptide which selectively binds to bone hydroxyapatite (HA). In another embodiment, the present disclosure provides for one or more bone-targeting polypeptides engineered to comprise at least one bone-homing peptide which selectively binds to bone HA.

In some aspects, the at least one bone-homing peptide is inserted at a permissive internal site of the antibody. In certain aspects, the at least one bone-homing peptide is inserted at the light chain (LC), heavy chain (CH1), and/or C-terminus (CT) of the antibody.

In some aspects, the at least one bone-homing peptide is inserted at the C-terminus or N-terminus of the one or more polypeptides.

In certain aspects, the antibody or polypeptide(s) comprises two, three, or four bone-homing peptides. In particular aspects, the bone-homing peptide is L-Asp₃, L-Asp₄, L-Asp₅, L-Asp₆, L-Asp₇, L-Aspx, L-Asp₉, or L-Asp₁₀. In some aspects, the bone-homing peptide comprises at least sequential three aspartic acids, such as at least four, five, six, seven, eight, nine or ten aspartic acids. In specific aspects, the bone-homing peptide is L-Asp₆.

In some aspects, the antibody is a monoclonal antibody, bispecific antibody, Fab′, a F(ab′)2, a F(ab′)3, a monovalent scFv, a bivalent scFv, a single domain antibody, or nanobody. In particular aspects, the antibody is an immune checkpoint inhibitor. In some aspects, the antibody is an anti-HER2 antibody, anti-CD99 antibody, anti-IGF-IR antibody, anti-PD-L1, anti-PD-1, anti-CTLA4-antibody, anti-Siglec-2 antibody, anti-Siglec-3 antibody, anti-Siglec-5 antibody, anti-Siglec-6 antibody, anti-Siglec-7 antibody, anti-Siglec-8 antibody, anti-Siglec9 antibody, anti-Siglec-10 antibody, anti-Siglec-11 antibody, anti-Siglec-15 antibody, anti-RANKL antibody, anti-EGFR antibody, anti-VEGFR antibody, anti-CD20 antibody, anti-CD22 antibody, anti-CD52 antibody, anti-Trop-2 antibody, anti-CD30 antibody, anti-CD152 antibody, anti-IL-6R antibody, anti-GD2 antibody, or anti-TGFβ antibody. In some aspects, the antibody is an anti-CD99 antibody.

In some aspects, the one or more polypeptides comprise an adrenergic agonist, an anti-apoptosis factor, an apoptosis inhibitor, a cytokine receptor, a cytokine, a cytotoxin, an erythropoietic agent, a glutamic acid decarboxylase, a glycoprotein, a growth factor, a growth factor receptor, a hormone, a hormone receptor, an interferon, an interleukin, an interleukin receptor, a kinase, a kinase inhibitor, a nerve growth factor, a netrin, a neuroactive peptide, a neuroactive peptide receptor, a neurogenic factor, a neurogenic factor receptor, a neuropilin, a neurotrophic factor, a neurotrophin, a neurotrophin receptor, an N-methyl-D-aspartate antagonist, a plexin, a protease, a protease inhibitor, a protein decarboxylase, a protein kinase, a protein kinase inhibitor, a proteolytic protein, a proteolytic protein inhibitor, a semaphorin, a semaphorin receptor, a serotonin transport protein, a serotonin uptake inhibitor, a serotonin receptor, a serpin, a serpin receptor, or a tumor suppressor. In particular aspects, the polypeptide comprises a cytokine. In specific aspects, the cytokine is IL-6. In particular aspects, the IL-6 comprises the bone-homing peptide at the C-terminus.

In further aspects, the antibody or polypeptide(s) is conjugated to a drug. In some aspects, the drug is monomethyl auristatin E (MMAE), ozogamicin, tiuxetan, vedotin, pasudotoxtdfx, vedotin, mafodotin, calicheamicin, maytansine, or deruxtecan. In particular aspects, the drug is an anti-mitotic drug, such as monomethyl auristatin E (MMAE).

In some aspects, the antibody is an anti-HER2 antibody. For example, the antibody is trastuzumab (Herceptin), pertuzumab (Perjeta), or atezolizumab. In particular aspects, the antibody is trastuzumab, such as trastuzumab is conjugated to MMAE. In specific aspects, the bone-homing peptide is inserted at residue A153, A165, and/or G449 of trastuzumab.

In certain aspects, the antibody has an amino acid sequence having at least 90% (e.g., 91%, 92%, 93%, 94%, 95%, 96%, 97%, 98%, or 99%) sequence identity to Tras-LC (SEQ ID NOs: 3-4), Tras-CH1 (SEQ ID NOs: 5-6), Tras-CT (SEQ ID NOs: 7-8), Tras-LC/CH1 (SEQ ID NOs: 9-10), Tras-LC/CT (SEQ ID NOs: 11-12), Tras-CH1/CT (SEQ ID NOs: 13-14), or Tras-LC/CH1/CT (SEQ ID NOs: 15-16). In particular aspects, the antibody comprises Tras-LC (SEQ ID NOs: 3-4), Tras-CH1 (SEQ ID NOs: 5-6), Tras-CT (SEQ ID NOs: 7-8), Tras-LC/CH1 (SEQ ID NOs: 9-10), Tras-LC/CT (SEQ ID NOs: 11-12), Tras-CH1/CT (SEQ ID NOs: 13-14), or Tras-LC/CH1/CT (SEQ ID NOs: 15-16).

In some aspects, the bone-targeting antibody or polypeptide(s) has increased binding affinity to HA as compared to an antibody or polypeptide(s) that does not comprise the bone-homing peptide. In particular aspects, the bone-targeting antibody has two-fold to three-fold higher binding affinity to HA as compared to an antibody or polypeptide(s) that does not comprise the bone-homing peptide.

A further embodiment provides a method of treating or preventing bone diseases (e.g., bone tumors) in a subject comprising administering to the subject an effective amount of a bone-targeting antibody or polypeptide(s) of any the present embodiments and aspects thereof (e.g., a bone-targeting antibody or polypeptide(s) engineered to comprise at least one bone-homing peptide which selectively binds to bone hydroxyapatite (HA)).

In some aspects, the subject has bone cancer or bone metastasis. In certain aspects, the bone cancer is Ewing sarcoma, osteosarcoma, or chondrosarcoma. In particular aspects, the bone metastasis is from breast cancer, myeloma, renal cancer, lung cancer, prostate cancer, thyroid cancer, or bladder cancer. In specific aspects, the breast cancer is triple-negative breast cancer, HER2-negative breast cancer, or HER2-positive breast cancer.

In certain aspects, the bone disease is osteoporosis, osteomalacia, periodontitis, rheumatoid arthritis, metabolic bone disease, a parathyroid disorder, steroid-induced osteoporosis, chemotherapy-induced bone loss, pre-menopausal bone loss, fragility and recurrent fractures, renal osteodystrophy, bone infections, or Paget's disease. The methods and compositions provided herein may be used to reduce cortical and/or trabecular bone loss, reduce cortical and/or trabecular bone mineral content loss, improve the bone biomechanical resistance, increase bone formation, and/or reduce bone-resorption.

In certain aspects, the bone-targeting antibody or polypeptide(s) results in increased concentration of therapeutic antibody or polypeptide(s) at the bone tumor niche, inhibits cancer development in the bone, and/or limits secondary metastases to other organs. In some aspects, the bone-targeting antibody or polypeptide(s) results in decreased micrometastasis-induced osteolyic lesions.

In further aspects, the method comprises further administering an additional anti-cancer therapy. In some aspects, the additional anti-cancer therapy comprises surgery, chemotherapy, radiation therapy, hormonal therapy, immunotherapy or cytokine therapy. For example, the additional anti-cancer therapy comprises immunotherapy or chemotherapy.

Another embodiment provides the use of a bone-targeting antibody or polypeptide(s) of any of the present embodiments and aspects thereof (e.g., a bone-targeting antibody engineered to comprise at least one bone-homing peptide which selectively binds to bone hydroxyapatite (HA)) for the treatment or prevention of bone disease (e.g, bone tumors) in a subject with cancer.

In some aspects, the subject has bone cancer or bone metastasis. In certain aspects, the bone cancer is Ewing sarcoma, osteosarcoma, or chondrosarcoma. In particular aspects, the bone metastasis is from breast cancer, myeloma, renal cancer, lung cancer, prostate cancer, thyroid cancer, or bladder cancer. In specific aspects, the breast cancer is triple-negative breast cancer, HER2-negative breast cancer, or HER2-positive breast cancer.

In certain aspects, the bone disease is osteoporosis, osteomalacia, periodontitis, rheumatoid arthritis, metabolic bone disease, a parathyroid disorder, steroid-induced osteoporosis, chemotherapy-induced bone loss, pre-menopausal bone loss, fragility and recurrent fractures, renal osteodystrophy, bone infections, or Paget's disease. The methods and compositions provided herein may be used to reduce cortical and/or trabecular bone loss, reduce cortical and/or trabecular bone mineral content loss, improve the bone biomechanical resistance, increase bone formation, and/or reduce bone-resorption.

In certain aspects, the bone-targeting antibody or polypeptide(s) results in increased concentration of therapeutic antibody or polypeptide(s) at the bone tumor niche, inhibits cancer development in the bone, and/or limits secondary metastases to other organs. In some aspects, the bone-targeting antibody results in decreased micrometastasis-induced osteolyic lesions.

In further aspects, the use further comprises an additional anti-cancer therapy. In some aspects, the additional anti-cancer therapy comprises surgery, chemotherapy, radiation therapy, hormonal therapy, immunotherapy or cytokine therapy. For example, the additional anti-cancer therapy comprises immunotherapy or chemotherapy.

Another embodiment provides a method for engineering a bone-targeting antibody or polypeptide(s) of any of the present embodiments and aspects thereof (e.g., a bone-targeting antibody or polypeptide(s) engineered to comprise at least one bone-homing peptide which selectively binds to bone hydroxyapatite (HA)) comprising inserting at least one bone-homing peptide at a permissive internal site of said antibody or at the C-terminus or N-terminus of said polypeptide(s).

In some aspects, the at least one bone-homing peptide is inserted at a permissive internal site of the antibody. In certain aspects, the at least one bone-homing peptide is inserted at the light chain (LC), heavy chain (CH1), and/or C-terminus (CT) of the antibody.

In certain aspects, the antibody or polypeptide(s) comprises two, three, or four bone-homing peptides. In particular aspects, the bone-homing peptide is L-Asp₃, L-Asp₄, L-Asp₅, L-Asp₆, L-Asp₇, L-Asp₈, L-Asp₉, or L-Asp₁₀. In some aspects, the bone-homing peptide comprises at least sequential three aspartic acids, such as at least four, five, six, seven, eight, nine or ten aspartic acids. In specific aspects, the bone-homing peptide is L-Asp₆.

In some aspects, the antibody is a monoclonal antibody, bispecific antibody, Fab′, a F(ab′)2, a F(ab′)3, a monovalent scFv, a bivalent scFv, a single domain antibody, or nanobody. In particular aspects, the antibody is an immune checkpoint inhibitor. In some aspects, the antibody is an anti-HER2 antibody, anti-CD99 antibody, anti-IGF-IR antibody, anti-PD-L1, anti-PD-1, anti-CTLA4-antibody, anti-Siglec-2 antibody, anti-Siglec-3 antibody, anti-Siglec-5 antibody, anti-Siglec-6 antibody, anti-Siglec-7 antibody, anti-Siglec-8 antibody, anti-Siglec9 antibody, anti-Siglec-10 antibody, anti-Siglec-11 antibody, anti-Siglec-15 antibody, anti-RANKL antibody, anti-EGFR antibody, anti-VEGFR antibody, anti-CD20 antibody, anti-CD22 antibody, anti-CD52 antibody, anti-Trop-2 antibody, anti-CD30 antibody, anti-CD152 antibody, anti-IL-6R antibody, anti-GD2 antibody, or anti-TGFβ antibody.

In some aspects, the one or more polypeptides comprise an adrenergic agonist, an anti-apoptosis factor, an apoptosis inhibitor, a cytokine receptor, a cytokine, a cytotoxin, an erythropoietic agent, a glutamic acid decarboxylase, a glycoprotein, a growth factor, a growth factor receptor, a hormone, a hormone receptor, an interferon, an interleukin, an interleukin receptor, a kinase, a kinase inhibitor, a nerve growth factor, a netrin, a neuroactive peptide, a neuroactive peptide receptor, a neurogenic factor, a neurogenic factor receptor, a neuropilin, a neurotrophic factor, a neurotrophin, a neurotrophin receptor, an N-methyl-D-aspartate antagonist, a plexin, a protease, a protease inhibitor, a protein decarboxylase, a protein kinase, a protein kinase inhibitor, a proteolytic protein, a proteolytic protein inhibitor, a semaphorin, a semaphorin receptor, a serotonin transport protein, a serotonin uptake inhibitor, a serotonin receptor, a serpin, a serpin receptor, or a tumor suppressor. In particular aspects, the polypeptide comprises a cytokine. In specific aspects, the cytokine is IL-6. In particular aspects, the IL-6 comprises the bone-homing peptide at the C-terminus.

In further aspects, the antibody or polypeptide(s) is conjugated to a drug. In some aspects, the drug is monomethyl auristatin E (MMAE), ozogamicin, tiuxetan, vedotin, pasudotoxtdfx, vedotin, mafodotin, calicheamicin, maytansine, or deruxtecan. In particular aspects, the drug is an anti-mitotic drug, such as monomethyl auristatin E (MMAE).

In some aspects, the antibody is an anti-HER2 antibody. For example, the antibody is trastuzumab (Herceptin), pertuzumab (Perjeta), or atezolizumab. In particular aspects, the antibody is trastuzumab, such as trastuzumab is conjugated to MMAE. In specific aspects, the bone-homing peptide is inserted at residue A153, A165, and/or G449 of trastuzumab.

In certain aspects, the antibody has an amino acid sequence having at least 90% (e.g., 91%, 92%, 93%, 94%, 95%, 96%, 97%, 98%, or 99%) sequence identity to Tras-LC (SEQ ID NOS: 3-4), Tras-CH1 (SEQ ID NOS: 5-6), Tras-CT (SEQ ID NOS: 7-8), Tras-LC/CH1 (SEQ ID NOS: 9-10), Tras-LC/CT (SEQ ID NOS: 11-12), Tras-CH1/CT (SEQ ID NOS: 13-14), or Tras-LC/CH1/CT (SEQ ID NOS: 15-16). In particular aspects, the antibody comprises Tras-LC (SEQ ID NOS: 3-4), Tras-CH1 (SEQ ID NOS: 5-6), Tras-CT (SEQ ID NOS: 7-8), Tras-LC/CH1 (SEQ ID NOS: 9-10), Tras-LC/CT (SEQ ID NOS: 11-12), Tras-CH1/CT (SEQ ID NOS: 13-14), or Tras-LC/CH1/CT (SEQ ID NOS: 15-16).

It is contemplated that any method or composition described herein can be implemented with respect to any other method or composition described herein. For example, a compound synthesized by one method may be used in the preparation of a final compound according to a different method.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1B: (FIG. 1A) Protein sequences of hydroxyapatite-binding proteins (SEQ ID NOS: 25-26). (FIG. 1B) Therapeutic antibodies can be specifically delivered to the bone by introducing bone-homing peptide sequence that bind to the bone hydroxyapatite matrix.

FIGS. 2A-2H: Preparation and characterization of bone-targeting antibodies. (FIG. 2A) The bone-homing peptide was inserted at three locations: light chain (LC), heavy chain (CH1), and c-terminus (CT). (FIG. 2B) SDS-PAGE analysis of bone-targeting antibodies in the presence (left) and absence (right) of the reducing reagents. (FIG. 2CC) Mass spectrometry analysis of bone-targeting antibodies. (FIG. 2D) Binding kinetics of Tras, Tras-CH1, Tras-LC, Tras-CT, Tras-LC/CT, Tras-CH1/CT and Tras-LC/CH1/CT to hydroxyapatite (HA). (FIG. 2E) Differential bone targeting ability of Tras and bone targeting conjugates. Non-decalcified bone sections from C57/BL6 mice were incubated with 50 μg/mL Tras or bone targeting conjugates overnight, followed by staining with fluorescein isothiocyanate (FITC)-labeled anti-human IgG and 4 μg/mL xylenol orange (XO, known to label bone), Scale bars, 200 μm. (FIG. 2F) Flow cytometric profiles of Tras, Tras-CT, Tras-CH1/CT, and Tras-LC/CH1/CT binding to SK-BR-3 (HER2+++) and MDA-MB-468 (HER2−) cells. (FIG. 2G) In vitro cytotoxicity of Tras, Tras-CT, Tras-CH1/CT, and Tras-LC/CH1/CT against SK-BR-3 and MDA-MB-468 cells. (FIG. 2H) Ex vivo fluorescence images of lower limbs of athymic nude mice bearing MDA-MB-361 tumors 72 h or 120 h after the retro-orbital injection of Cy7.5-labeled Tras, Tras-CT, Tras-CH1/CT, and Tras-LC/CH1/CT antibodies. Tumor cells were inoculated into the right tibiae of nude mice via para-tibial injection.

FIGS. 3A-3S: Bone-targeting antibodies inhibit breast cancer bone metastases. (FIG. 3A) MDA-MB-361 cells were para-tibia injected into the right hind limb of nude mice, followed by treatment with PBS, Tras (1 mg/kg retro-orbital venous sinus in sterile PBS twice a week), Tras-CT, Tras-CH1/CT, and Tras-LC/CH1/CT (same as Tras). Tumor burden was monitored by weekly bioluminescence imaging. (FIGS. 3B-3C) Fold-change in mean luminescent intensity of MDA-MB-361 tumors in mice treated as described in FIG. 3A. p values are based on two-way ANOVA test. (FIG. 3D) Kaplan-Meier plot of the time-to-euthanasia of mice treated as described in FIG. 3A. For each individual mouse, the BLI signal in the whole body reached 10⁷ photons sec⁻¹ was considered as the endpoint. (FIG. 3E) Body weight change of tumor-bearing mice over time. (FIG. 3F) MicroCT scanning in the supine position for groups treated with PBS, Tras, Tras-CT, Tras-CH1/CT, and Tras-LC/CH1/CT. (FIG. 3G) Quantitative analysis of bone volume (BV). (FIG. 3H) Quantitative analysis of bone surface/bone volume ratio (BS/BV). (FIG. 3I) Quantitative analysis of bone volume/tissue volume ratio (BV/TV). (FIG. 3J) Quantitative analysis of trabecular thickness (Tb.Th). (FIG. 3K) Quantitative analysis of trabecular bone mineral density (BMD). (FIG. 3L) Representative longitudinal, midsagittal hematoxylin and eosin (H&E)-stained sections of tibia/femur from each group. T: tumor; B: bone; BM: bone marrow. (FIG. 3M) Representative images of HER2 and TRAP staining of bone sections from each group. (FIG. 3N) Osteoclast number per image calculated at the tumor-bone interface in each group (pink cells in FIG. 3K were considered as osteoclast positive cells). (FIG. 3O) MDA-MB-361 cells were para-tibia injected into the right hind limb of nude mice, followed by treatment with Tras (10 mg/kg retro-orbital venous sinus in sterile PBS every 2 weeks for two months) and Tras-CH1/CT (same as Tras). Tumor burden was monitored by weekly bioluminescence imaging. (FIG. 3P) Fold-change in mean luminescent intensity of MDA-MB-361 tumors in mice treated as described in FIG. 3O. (FIG. 3Q) Fold-change in individual luminescent intensity of MDA-MB-361 tumors in mice treated as described in (O). (FIG. 3R) Kaplan-Meier plot of the time-to-euthanasia of mice treated as described in FIG. 3O. For each individual mouse, the BLI signal in the whole body reached 10⁸ photons s⁻¹ was considered as the end point. (FIG. 3S) Body weight change of tumor-bearing mice in FIG. 30 over time. ****P<0.0001, ***P<0.001, **P<0.01, *P<0.05, and n.s. P>0.05.

FIGS. 4A-4C: (FIG. 4A) Bone lesions more readily give rise to secondary metastases to multiple organs. (FIG. 4B) Secondary metastases observed in various organs in mice treated with Tras or Tras-CH1/CT. (FIG. 4C) Heat map of ex vivo BLI intensity and status of metastatic involvement in tissues from mice treated with PBS, Tras, Tras-CT, Tras-CH1/CT, and Tras-LC/CH1/CT. Each column represents an individual animal, and each row represents a type of tissue. The presence of the metastasis was defined as the presence of BLI signal above 18 counts/pixel under 120 seconds exposure time. Multi-site metastases were defined as the metastatic involvement of at least three tissues. p values were determined by Fisher's exact test on the frequency of metastatic involvement while by Mann-Whitney test of on the metastatic burden.

FIG. 5: ESI-MS spectra of Tras.

FIG. 6: ESI-MS spectra of Tras-LC.

FIG. 7: ESI-MS spectra of Tras-CH1.

FIG. 8: ESI-MS spectra of Tras-CT.

FIG. 9: ESI-MS spectra of Tras-LC/CH1.

FIG. 10: ESI-MS spectra of Tras-LC/CT.

FIG. 11: ESI-MS spectra of Tras-CH1/CT.

FIG. 12: ESI-MS spectra of Tras-LC/CH1/CT.

FIG. 13: Differential trabecular bone targeting ability of Tras, Tras-CT, Tras-CH1/CT and Tras-LC/CH1/CT. Non-decalcified bone sections from C57/BL6 mice were incubated with 50 g/mL Tras, Tras-CT, Tras-CH1/CT or Tras-LC/CH1/CT overnight, followed by staining with fluorescein isothiocyanate (FITC)-labeled anti-human IgG and 4 μg/mL xylenol orange (XO, known to label bone). Scale bars, 200 μm.

FIG. 14: Tras binding to SK-BR-3 cells. SK-BR-3 cells were incubated with increasing concentrations of Tras and fluorescence was measured on the flow cytometer. The KD was determined as follows: 1/F=1/Fmax+(KD/Fmax) (1/[Ab].

FIG. 15: Tras-LC binding to SK-BR-3 cells. SK-BR-3 cells were incubated with increasing concentrations of Tras-LC and fluorescence was measured on the flow cytometer. The KD was determined as follows: 1/F=1/Fmax+(KD/Fmax) (1/[Ab].

FIG. 16: Tras-CT binding to SK-BR-3 cells. SK-BR-3 cells were incubated with increasing concentrations of Tras-CT and fluorescence was measured on the flow cytometer. The KD was determined as follows: 1/F=1/Fmax+(KD/Fmax) (1/[Ab].

FIG. 17: Tras-CH1 binding to SK-BR-3 cells. SK-BR-3 cells were incubated with increasing concentrations of Tras-CH1 and fluorescence was measured on the flow cytometer. The KD was determined as follows: 1/F=1/Fmax+(KD/Fmax) (1/[Ab].

FIG. 18: Tras-LC/CT binding to SK-BR-3 cells. SK-BR-3 cells were incubated with increasing concentrations of Tras-LC/CT and fluorescence was measured on the flow cytometer. The KD was determined as follows: 1/F=1/Fmax+(KD/Fmax) (1/[Ab].

FIG. 19: Tras-CH1/CT binding to SK-BR-3 cells. SK-BR-3 cells were incubated with increasing concentrations of Tras-CH1/CT and fluorescence was measured on the flow cytometer. The KD was determined as follows: 1/F=1/Fmax+(KD/Fmax) (1/[Ab].

FIG. 20: Tras-CH1/CT binding to SK-BR-3 cells. SK-BR-3 cells were incubated with increasing concentrations of Tras-CH1/CT and fluorescence was measured on the flow cytometer. The KD was determined as follows: 1/F=1/Fmax+(KD/Fmax) (1/[Ab].

FIG. 21: Tras-LC/CH1 binding to SK-BR-3 cells. SK-BR-3 cells were incubated with increasing concentrations of Tras-LC/CH1 and fluorescence was measured on the flow cytometer. The KD was determined as follows: 1/F=1/Fmax+(KD/Fmax) (1/[Ab].

FIG. 22: Tras-LC/CH1/CT binding to SK-BR-3 cells. SK-BR-3 cells were incubated with increasing concentrations of Tras and fluorescence was measured on the flow cytometer. The KD was determined as follows: 1/F=1/Fmax+(KD/Fmax) (1/[Ab].

FIG. 23: Cell-Surface binding of Tras-CH1, Tras-LC, Tras-LC/CT and Tras-LC/CH1 against SK-BR-3 and MDA-MB-468 cells. Flow cytometric profiles of Tras-CH1, Tras-LC, Tras-LC/CT and Tras-LC/CH1 binding to SK-BR-3 (HER2+++) and MDA-MB-468 (HER2−) cells.

FIG. 24: Ex vivo fluorescence images of main organs. Heart, liver, spleen, lung, kidney, brain of C57/BL6 mice 48 h after the retro-orbital injection of Cy7.5-labeled Tras, Tras-CT and Tras-LC/CH1/CT.

FIGS. 25A-25C: In vivo and ex vivo fluorescence images analysis for the biodistribution of Tras, Tras-CT, Tras-CH1/CT and Tras-LC/CH1/CT. (FIG. 25A) 72 h or 120 h after the retro-orbital injection of Cy7.5-labeled Tras, Tras-CT, Tras-CH1/CT and

Tras-LC/CH1/CT. The bone and main organs (heart, liver, spleen, lung, kidney, brain) were collected and analysis. (FIG. 25B) Quantitative analysis of Tras and bone-targeting antibody distributions in different tissues (heart, liver, spleen, lung, kidney, brain and bone. (FIG. 25C) Histological analysis of heart with H&E staining after treatments with PBS, Tras-CT, Tras-CH1/CT, and Tras-LC/CH1/CT. *P<0.05, and n.s. P>0.05.

FIGS. 26A-26C: Fold change of BLI signal intensity during the treatment. MDA-MB-361 cells were para-tibia injected into the right hind limb of nude mice, followed by treatment with PBS, Tras (1 mg/kg retro-orbital venous sinus in sterile PBS twice a week), Tras-CT, Tras-CH1/CT and Tras-LC/CH1/CT (same as Tras). Tumor burden was monitored by weekly bioluminescence imaging. (FIG. 26A) Fold-change in mean luminescent intensity of MDA-MB-361 tumors in mice treated as described in FIG. 3A, two-way ANOVA comparing Tras, Tras-CT, Tras-CH1/CT and Tras-LC/CH1/CT groups. (FIG. 26B) two-way ANOVA comparing fold-change in mean luminescent intensity between Tras-CT, Tras-CH1/CT and Tras-LC/CH1/CT groups. (FIG. 26C) Two-way ANOVA comparing fold-change in individual luminescent intensity between Tras-CT, Tras-CH1/CT and Tras-LC/CH1/CT.

FIGS. 27A-27D: BLI signaling quantification. (FIG. 27A) The BLI from each treatment group quantified by the radiance detected in the region of interest. (FIG. 27B) Two-way ANOVA comparing BLI between Tras, Tras-CT, Tras-CH1/CT and Tras-LC/CH1/CT groups. (FIG. 27C) two-way ANOVA comparing BLI between Tras-CT, Tras-CH1/CT and Tras-LC/CH1/CT groups. (FIG. 27D) Individual luminescent intensity of different treated group as described in FIG. 27C. **P<0.01.

FIG. 28: MicroCT-based 3D renderings of bones. Cortical bone, images of the cortical compartment show cortical bone destruction of Con, Tras, Tras-CT, Tras-CH1/CT and Tras-CH1/CL/CT treatment groups. Trabecular bone, images of the cortical compartment show trabecular bone destruction of Con, Tras, Tras-CT, Tras-CH1/CT and Tras-CH1/CL/CT treatment groups.

FIG. 29: Representative microCT slices from each treatment groups. MicroCT slices near the growth plate (lower panel), 1.25 mm distal of the growth plate (middle panl, consider the trabecular bone), and 3.25 mm distal of the growth plate (middle plate, consider the cortical bone).

FIG. 30: Representative images of HER2 staining of bone sections from each group.

FIG. 31: TRAP staining of bone sections from Control and Tras groups.

FIG. 32: TRAP staining of bone sections from Tras-CT, Tras-CH1/CT and Tras-LC/CH1/CT.

FIGS. 33A-33B: Effects of bone-targering antibodies on MDA-MB-361 model: serum TRACP 5b and calcium levels analysis. (FIG. 33A) Serum TRAcP 5b levels of mice treated with PBS, Tras, Tras-CT, Tras-CH1/CT and Tras-LC/CH1/CT. (FIG. 33B) Serum calcium levels of mice from each treatment groups. **P<0.01, *P<0.05, and n.s. P>0.05.

FIGS. 34A-34B: Bone-targeting antibodies effect on multi-organs metastases in MDA-MB-361 cell lines. (FIG. 34A) Secondary metastases observed in various organs in mice treated with PBS, Tras-CT or Tras-LC/CH1/CT. (FIG. 34B) Heat map of ex vivo BLI intensity and status of metastatic involvement in tissues from mice treated with PBS, Tras, Tras-CT and Tras-LC/CH1/CT. Each column represents an individual animal, and each row represents a type of tissue. The presence of the metastasis was defined as the presence of BLI signal above 18 counts/pixel under 120 second exposure time. Multi-site metastases was defined as the metastatic involvement of at least three tissues. p value were determined by Fisher's exact test on the frequency of metastatic involvement while by Mann-Whitney test of on the metastatic burden.

FIG. 35: Antibody-drug conjugate Trastuzumab-monomethyl auristatin E modified with bone-homing peptide ADC demonstrated better anti-tumor activity.

FIGS. 36A-36E: (FIG. 36A) MCF-7 cells were para-tibia injected into the right hind limb of nude mice, followed by treatment with Tras (1 mg/kg retro-orbital venous sinus in sterile PBS twice a week for two months) and Tras-CH1/CT (same as Tras). Tumor burden was monitored by weekly bioluminescence imaging. (FIG. 36B) Fold-change in mean luminescent intensity of MCF-7 tumors in mice treated as described in FIG. 36A. p values are based on a two-way ANOVA test. (FIG. 36C) Fold-change in individual luminescent intensity of MCF-7 tumors in mice treated as described in FIG. 36A. (FIG. 36D) Kaplan-Meier plot of the time-to-euthanasia of mice treated as described in FIG. 36A. For each individual mouse, the BLI signal in the whole body reaching 5×10⁷ photons s⁻¹ was considered the end point. (FIG. 36E) Body weight change of tumor-bearing mice in FIG. 36A over time. ****P<0.0001, *P<0.05, and n.s. P>0.05.

FIGS. 37A-37K: (FIG. 37A) Preparation of bone-targeting antibody-drug conjugates. Tras antibody was first modified with the bone-homing peptide at the heavy chain (CH1) and c-terminus (CT), followed by the modification of MMAE using pClick antibody conjugation technology. (FIG. 37B) SDS-PAGE analysis of Tras-MMAE and Tras-CH1/CT-MMAE under nonreducing (left) and reducing (right) conditions. (FIGS. 37C,D) In vitro cytotoxicity of MMAE, Tras-MMAE, and Tras-CH1/CT-MMAE, against SK-BR-3 and MDA-MB-468 cancer cells. (FIG. 37E) Differential bone targeting ability of Tras-MMAE and Tras-CH1/CT-MMAE. Non-decalcified bone sections from C57/BL6 mice were incubated with 50 μg/mL Tras-MMAE and Tras-CH1/CT-MMAE overnight, followed by staining with fluorescein isothiocyanate (FITC)-labeled anti-human IgG and 4 μg/mL xylenol orange (XO, known to label bone). (FIG. 37F) MDA-MB-361 cells were para-tibia injected into the right hind limb of nude mice, followed by treatment with Tras-MMAE (0.5 mg/kg retro-orbital venous sinus in sterile PBS every week for two months) and Tras-CH1/CT-MMAE (same as Tras). Tumor burden was monitored by weekly bioluminescence imaging. (FIG. 37G) Fold-change in mean luminescent intensity of MDA-MB-361 tumors in mice treated as described in FIG. 37F. (FIG. 37H) Fold-change in individual luminescent intensity of MDA-MB-361 tumors in mice treated as described in FIG. 37F. (FIG. 37I) Body weight change of tumor-bearing mice in FIG. 37F over time. (FIG. 37J) Micro-CT scanning of bones from mice treated with Tras-MMAE and Tras-CH1/CT-MMAE after tumor implantation. (FIG. 37K) Heat map of ex vivo BLI intensity and status of metastatic involvement in tissues from mice treated with Tras-MMAE and Tras-CH1/CT-MMAE. Each column represents an individual animal, and each row represents a type of tissue. The presence of the metastasis was defined as the presence of BLI signal above 18 counts/pixel under 120 s exposure time. Multisite metastases were defined as the metastatic involvement of at least three tissues. p Values were determined by Fisher's exact test on the frequency of metastatic involvement and by the Mann-Whitney test of on the metastatic burden. ****P<0.0001 and n.s. P>0.05.

FIGS. 38A-38B: Safety evaluation of Tras and Tras-CH1/CT. (FIG. 38A) RAW264.7 and (FIG. 38B) MC3T3-E1 cells.

FIGS. 39A-39G: Analysis of tumor burden at bone site. (FIG. 39A) MicroCT scanning in the supine position for groups treated with PBS, Tras, Tras-CT, Tras-CH1/CT, Tras-LC/CH1/CT and Sham. (FIG. 39B) Quantitative analysis of bone volume (BV). (FIG. 39C) Quantitative analysis of bone volume/tissue volume ratio (BV/TV). (FIG. 39D) Quantitative analysis of trabecular bone mineral density (BMD). (FIG. 39E) Quantitative analysis of trabecular thickness (Tb.Th). (FIG. 39F) Quantitative analysis of bone surface/bone volume ratio (BS/BV). ***P<0.001, **P<0.01, *P<0.05, and n.s. P>0.05. (FIG. 39G) Representative longitudinal and midsagittal H&E stained sections of tibia/femur from each group. T: tumor; B: bone; BM: bone marrow.

FIGS. 40A-40B: In vitro stability of Tras-CH1/CT. (FIG. 40A) ESI-MS spectra of Tras-CH1/CT after incubating in PBS at 4° C. for 3 months. (FIG. 40B) SDS-PAGE analysis of Tras and Tras-CH1/CT after incubating in PBS at 4° C. for 3 months.

FIG. 41: Pharmacokinetic profiles of Tras and Tras-CH1/CT. Tumor bearing athymic nude mice (3 months after surgery) were injected retro-orbitally with 1 mg/kg Tras and Tras-CH1/CT in PBS. Antibody concentrations in the serum were determined by ELISA kit (Data was presented as mean±SD for three independent repeats).

FIG. 42: High dose of bone-targeting antibodies inhibit breast cancer bone metastases. Tumor burden was monitored by weekly BLI.

FIGS. 43A-43B: BLI signaling quantification from high dose in MDA-MB-361 treatment groups. (FIG. 43A) The BLI from each treatment group quantified by the radiance detected in the region of interest. two-way ANOVA comparing BLI between Tras and Tras-CH1/CT groups. (FIG. 43B) Individual luminescent intensity of different treated group as described in FIG. 30. ****P<0.0001.

FIGS. 44A-44B: BLI signaling quantification from treatment groups of MCF-7 model. (FIG. 44A) The BLI from each treatment group quantified by the radiance detected in the region of interest. two-way ANOVA comparing BLI between Tras and Tras-CH1/CT groups. (FIG. 44B) Individual luminescent intensity of different treated group as described in FIGS. 4A-C. ****P<0.0001.

FIGS. 45A-45C: Therapeutic effect of Tras-CH1/CT on the mice with primary tumor. (FIG. 45A) Surgically removed tumor tissues from nude mice 21 days after inoculation. (FIG. 45B) Tumor volumes of mice treated with PBS, Tras or Tras-CH1/CT at different time-points after inoculation. (FIG. 45C) Tumor volumes of mice treated with Tras or Tras-CH1/CT at different timepoints after inoculation. n.s. P>0.05.

FIGS. 46A-46E: Evaluation of the immune response of mice after treatment with Tras-CH1/CT. (FIG. 46A) B cells, monocytes, neutrophils, macrophages, CD4+ T cells, and CD8+ T cells suspension obtained from blood were stained for various surface markers and analyzed by flow cytometry. (FIG. 46B) IFNγ levels in CD4+ T, CD8+ T, and B cells. (FIGS. 46C, 46D, 46E) IL2, IL4 and IFNγ analysis of plasma samples from mice. n.s. P>0.05.

FIG. 47: Anti-trastuzumab antibody levels developed in mice injected with Tras and Tras-CH1/CT. Tras and Tras-CH1/CT groups: mice were treated with Tras and Tras-CH1/CT (5 mg/kg), twice a week for one month.

FIG. 48: ESI-MS spectra of Tras-MMAE.

FIG. 49: ESI-MS spectra of Tras-CH1/CT-MMAE.

FIG. 50: Cell-Surface binding of Tras-MMAE and Tras-CH1/CT-MMAE against SKBR-3 and MDA-MB-468 cells. Cells were incubated with 30 nM Tras-ALN in media for 30 min at 37° C. and Hoechst nuclear stain (blue fluorescence).

FIGS. 51A-51B: Fold change of BLI signal intensity during Tras-CH1/CT-MMAE treatment. (FIG. 51A) Fold-change in mean luminescent intensity of MDA-MB-361 tumors in mice treated as described in FIG. 37F, two-way ANOVA comparing Tras-MMAE and Tras-CH1/CT-MMAE groups. (FIG. 51B) two-way ANOVA comparing fold-change in individual luminescent intensity between Tras-MMAE and Tras-CH1/CT-MMAE. ****P<0.0001.

FIGS. 52A-52D: BLI signaling quantification from Tras-CH1/CT-MMAE treated groups. (FIG. 52A) The BLI from each treatment group quantified by the radiance detected in the region of interest. Two-way ANOVA comparing BLI between PBS, Tras-MMAE and Tras-CH1/CTMMAE groups. (FIG. 52B) Individual luminescent intensity of PBS, Tras-MMAE and Tras-CH1/CT-MMAE treated groups. (FIG. 52C) Two-way ANOVA comparing BLI between Tras-MMAE and Tras-CH1/CT-MMAE groups. (FIG. 52D) Individual luminescent intensity of Tras-MMAE and Tras-CH1/CT-MMAE treated groups. ****P<0.0001.

FIG. 53: Representative microCT slices of cortical bone and trabecular bone from each treatment groups.

FIGS. 54A-54B: Effects of Tras-CH1/CT-MMAE on MDA-MB-361 model: BV/TV and Tb.Th analysis. (FIG. 54A) Quantitative analysis of bone volume/tissue volume ratio (BV/TV). (FIG. 54B) Quantitative analysis of trabecular thickness (Tb.Th). **P<0.01 and n.s. P>0.05.

FIGS. 55A-55B: Comparation of Tras-CH1/CT-MMAE treated group with other treatment groups. (FIG. 55A) Representative longitudinal, midsagittal hematoxylin and eosin (H&E)-stained sections of tibia/femur from each group. (FIG. 55B) Representative images of HER2 staining of bone sections from each group.

FIG. 56: Secondary metastases observed in various organs in mice treated with Tras-MMAE or Tras-CH1/CT-MMAE.

FIGS. 57A-57D: Preparation and characterization of bone-targeting antibodies. (FIG. 57A) The bone-homing peptide was inserted at three locations: light chain (LC), heavy chain (CH1), and c-terminus (CT). (FIG. 57B) ESI-MS analysis of the antibodies. (FIG. 57C) Binding of αCD99 antibody to Ewing sarcoma and osteosarcoma cell lines. (FIG. 57D) Cell apoptosis induced by αCD99 antibody in ES cells detected by flow cytometry.

FIGS. 58A-58D: (FIG. 58A) Binding kinetics of αCD99 antibodies on hydroxyapatite (HA). (FIG. 58B) Differential bone targeting ability of wild type αCD99 and bone-targeting αCD99 mutants. Scale bars, 200 μm. (FIG. 58C) Ex vivo fluorescence images of lower limbs of athymic nude mice bearing SK-ES-1 tumor 7d or 9d after the retro-orbital injection NIR-labeled αCD99, and αCD99-CH1/CT. (FIGS. 58D-58E) In vitro cytotoxicity of αCD99 against HUVEC cells and monocytes.

FIGS. 59A-59D: 12E7 antibody inhibits Ewing sarcoma tumors in xenograft models. (FIG. 59A) SK-ES-1 cells were intra-tibia injected into the right hind limb of nude mice, followed by treatment with PBS and 12E7 antibody (1 mg/kg retro-orbital venous sinus in sterile PBS twice a week). Tumor burden was monitored by weekly bioluminescence imaging. (FIG. 59B) Signal progression of Fluc activity in mice treated as described in FIG. 59A) p values are based on a two-way ANOVA test. (FIG. 59C) Body weights of mice treated as described in FIG. 59A. (FIG. 59D) SK-ES-1 cells were intramuscularly injected in close proximity to the tibia of nude mice, followed by treatment with PBS and 12E7 antibody (1 mg/kg retro-orbital venous sinus in sterile PBS twice a week). (FIG. 59E) Signal progression of Fluc activity in mice treated as described in FIG. 59D. p values are based on a two-way ANOVA test. (FIG. 59F) Body weights of mice treated as described in FIG. 59D. ****P<0.0001, ***P<0.001, **P<0.01, *P<0.05, ns>0.05.

FIG. 60: Pulmonary and skeletal metastases observed in various organs in mice following the establishment of intra tibia-introduced bone lesions in right legs.

FIGS. 61A-61B: (FIG. 61A) Expression and purification of IL6-6D. (FIG. 61B) Binding kinetics of IL6-6D on hydroxyapatite.

### DESCRIPTION OF ILLUSTRATIVE EMBODIMENTS

Therapeutic antibodies have gone a long way toward realizing their clinical potential and have become useful for treating a variety of pathologies. Despite the rapid evolution of therapeutic antibodies, their clinical efficacy in treatment of bone tumors has been hampered by the inadequate pharmacokinetics and poor bone tissue accessibility of these large macromolecules. Accordingly, in certain embodiment, the present disclosure, provides methods for engineering therapeutic antibodies or polypeptide(s) to include bone-homing peptide sequences that enhances their concentration in the bone metastatic niche, resulting in significantly reduced survival and progression of breast cancer bone metastases. To enhance the bone tumor-targeting ability of engineered antibodies, varying numbers of a bone-homing peptide may be introduced into permissive internal sites of the antibody. Compared to the unmodified antibody, the engineered bone-targeting antibodies were shown to have similar pharmacokinetics and in vitro cytotoxic activity but exhibited improved bone tumor distribution in vivo. These results demonstrate that adding bone-specific targeting to antibody therapy results in robust delivery of therapeutic antibodies to the bone tumor niche. This provides a powerful strategy for overcoming inadequate treatment of bone cancer and the development of acquired resistance to therapy.

Accordingly, in certain embodiments, the present disclosure provides an innovative bone targeting technology that enables the specific delivery of therapeutic antibodies or polypeptide(s) to the bone via engineering of the antibodies or polypeptide(s) to comprise bone-homing peptides. This type of specific delivery of therapeutic antibodies or polypeptide(s) to the bone has the potential to enhance both the breadth and potency of antibody therapy for bone-related diseases.

### I. Definitions

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “about” means, in general, within a standard deviation of the stated value as determined using a standard analytical technique for measuring the stated value. The terms can also be used by referring to plus or minus 5% of the stated value.

The phrase “effective amount” or “therapeutically effective” means a dosage of a drug or agent sufficient to produce a desired result. The desired result can be subjective or objective improvement in the recipient of the dosage, increased lung growth, increased lung repair, reduced tissue edema, increased DNA repair, decreased apoptosis, a decrease in tumor size, a decrease in the rate of growth of cancer cells, a decrease in metastasis, or any combination of the above.

As used herein, the term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class, including any of the human classes: IgG, IgM, IgA, IgD, and IgE. The antibody may be a bi-specific antibody. In exemplary embodiments, antibodies used with the methods and compositions described herein are derivatives of the IgG class. The term antibody also refers to antigen-binding antibody fragments. Examples of such antibody fragments include, but are not limited to, Fab, Fabÿ, F(abÿ)2, scFv, Fv, dsFv diabody, and Fd fragments. Antibody fragment may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 10 amino acids and more typically will comprise at least about 200 amino acids.

“Subject” and “patient” refer to either a human or non-human, such as primates, mammals, and vertebrates. In particular embodiments, the subject is a human.

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of a tumor or malignancy, delay or slowing of tumor growth and/or metastasis, and an increased lifespan as compared to that expected in the absence of treatment.

### II. Bone-Targeting Antibody

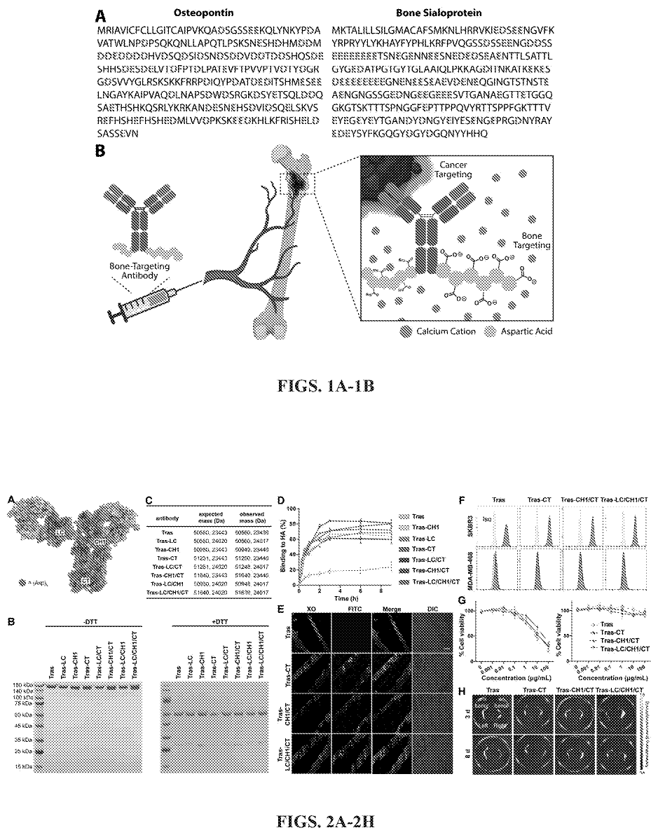

Bones are composed primarily of hydroxyapatite (HA) crystals, the insoluble salts of calcium and phosphorus. The restricted distribution of HA in hard tissues such as bone makes it an attractive target for selective bone targeting. Nature has evolved a variety of HA-binding proteins, including sialoprotein and osteopontin, that provide sites for cell anchorage and for modulating the bone mineralization process. Interestingly, sequence analysis reveals that these proteins have repeating sequences of acidic amino acids that represent possible bone binding sites (FIG. 1A).¹⁷ Short bone-homing peptides consisting of aspartic acid (Asp) have been tested for specific delivery of small molecules, microRNAs, and nanoparticles to the bone niche.¹⁸⁻²² These short peptides have been shown to favor binding to HA surface with higher levels of crystallinity. This surface is characterized by the presence of bone resorption surfaces and are known as the osteolytic bone metastatic niche.²³ Formation of osteolytic bone lesions is driven by paracrine crosstalk among cancer cells, osteoblasts, and osteoclasts.²²⁻²⁶ Specifically, cancer cells secrete molecules such as parathyroid hormone-related protein (PTHrP) and interleukin 8 that stimulate osteoclast formation directly or indirectly by acting to modulate the expression of osteoblast genes such as receptor activator of nuclear factor-KB ligand (RANKL) and osteoprotegerin (OPG). The consequent increase in bone resorption leads to release of growth factors (e.g., IGF1) that reciprocally stimulate tumor growth. Thus, selective delivery of therapeutic agents to the bone metastatic niche has the potential to interrupt this vicious osteolytic cycle. Accordingly, methods are provided herein for engineering antibodies capable of enhanced targeting of bone tumors. Following the insertion of bone-homing peptide sequences into anti-tumor antibodies, the utility of these engineered antibodies was demonstrated for the treatment of breast cancer metastases in bone (FIG. 1B).

In certain embodiments, provided herein are methods and compositions concerning bone-targeting antibodies, such as antibodies engineered to comprise one or more bone-homing peptides. In some aspects, the bone-homing peptide is a peptide which binds to bone hydroxyapatite (HA) matrix, such as L-Asp₆.

The bone-homing peptide(s) may be inserted in the antibody at a “permissive internal site” referred to herein as a site wherein insertion of a peptide is minimally disruptive to the native IgG structure and function, yet allows retention of the peptide's high affinity for bone matrix. To screen for permissive internal sites, flow cytometry may be used to ensure it will not disrupt the antibody function. The binding affinity of the bone-targeting antibody can be evaluated using an HA binding assay. The bone-homing peptide may be inserted at the light chain (LC), heavy chain (CH1), and/or C-terminus (CT) of the antibody. For example, the antibody may be trastuzumab. In specific aspects, the antibody may comprise protein sequences of SEQ ID NOs: 3-16 or protein sequences having at least 80%, 85%, 90%, 95%, 96%, 97%, 98%, or 99% sequence identity to SEQ ID NOs: 3-16. Exemplary amino acid sequences of unmodified and modified trastuzumab are provided below with the bone-homing peptide underlined.

Tras
Light chain (SEQ ID NO: 1):
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTVAAPSVF
IFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
Heavy chain (SEQ ID NO: 2):
EVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYW
GQGTLVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKSCD
KTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSLSLSPG
Tras-LC
Light chain (SEQ ID NO: 3):
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTVAAPSVF
IFPPSDEQLKSGTASVVCLLNNFYPREAKVQWKVDNADDDDDDQSGNSQESVTEQD
SKDSTYSLSSTLTLSKADYEKHKVYACEVTHQGLSSPVTKSFNRGEC
Heavy chain (SEQ ID NO: 4):
EVQLVESGGGLVQPGGSLRLSCAASGFNIKDTYIHWVRQAPGKGLEWVARIYPING
YTRYADSVKGRFTISADTSKNTAYLQMNSLRAEDTAVYYCSRWGGDGFYAMDYW
GQGTLVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKSCD
KTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSLSLSPG
Tras-CH1
Light chain (SEQ ID NO: 5):
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTVAAPSVF
IFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
Heavy chain (SEQ ID NO: 6):
EVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYW
GQGTLVTVSSASTKGPSVFPLAPSSKSTSGGTAALGCLVKDYFPEPVTVSWNSGADD
DDDDGVHTFPAVLQSSGLYSLSSVVTVPSSSLGTQTYICNVNHKPSNTKVDKKVEPK
SCDKTHTCPPCPAPELLGGPSVFLFPPKPKDTLMISRTPEVTCVVVDVSHEDPEVKEN
WYVDGVEVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLS
PGVEPKSCDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVS
NKALPAPIEKTISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHEALHNHYTQ
KSLSLSPG
Tras-CT
Light chain (SEQ ID NO: 7):
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTVAAPSVF
IFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
Heavy chain (SEQ ID NO: 8):
EVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYW
GQGTLVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKSCD
KTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSLSLSPGD
DDDDD
Tras-LC/CH1
Light chain (SEQ ID NO: 9):
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTVAAPSVF
IFPPSDEQLKSGTASVVCLLNNFYPREAKVQWKVDNADDDDDDQSGNSQESVTEQD
SKDSTYSLSSTLTLSKADYEKHKVYACEVTHQGLSSPVTKSFNRGEC
Heavy chain (SEQ ID NO: 10):
EVQLVESGGGLVQPGGSLRLSCAASGFNIKDTYIHWVRQAPGKGLEWVARIYPING
YTRYADSVKGRFTISADTSKNTAYLQMNSLRAEDTAVYYCSRWGGDGFYAMDYW
GQGTLVTVSSASTKGPSVFPLAPSSKSTSGGTAALGCLVKDYFPEPVTVSWNSGADD
DDDDGVHTFPAVLQSSGLYSLSSVVTVPSSSLGTQTYICNVNHKPSNTKVDKKVEPK
SCDKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLS
PGVEPKSCDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVS
NKALPAPIEKTISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHEALHNHYTQ
KSLSLSPG
Tras-LC/CT
Light chain (SEQ ID NO: 11):
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTVAAPSVF
IFPPSDEQLKSGTASVVCLLNNFYPREAKVQWKVDNADDDDDDQSGNSQESVTEQD
SKDSTYSLSSTLTLSKADYEKHKVYACEVTHQGLSSPVTKSFNRGEC
Heavy chain (SEQ ID NO: 12):
EVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYW
GQGTLVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKSCD
KTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSLSLSPGD
DDDDD
Tras-CH1/CT
Light chain (SEQ ID NO: 13):
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTVAAPSVF
IFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
Heavy chain (SEQ ID NO: 14):
EVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYW
GQGTLVTVSSASTKGPSVFPLAPSSKSTSGGTAALGCLVKDYFPEPVTVSWNSGADD
DDDDGVHTFPAVLQSSGLYSLSSVVTVPSSSLGTQTYICNVNHKPSNTKVDKKVEPK
SCDKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLS
PGVEPKSCDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVS
NKALPAPIEKTISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHEALHNHYTQ
KSLSLSPGQDI
Tras-LC/CH1/CT
Light chain (SEQ ID NO: 15):
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTVAAPSVF
IFPPSDEQLKSGTASVVCLLNNFYPREAKVQWKVDNADDDDDDQSGNSQESVTEQD
SKDSTYSLSSTLTLSKADYEKHKVYACEVTHQGLSSPVTKSFNRGEC
Heavy chain (SEQ ID NO: 16):
EVQLVESGGGLVQPGGSLRLSCAASGFNIKDTYIHWVRQAPGKGLEWVARIYPING
YTRYADSVKGRFTISADTSKNTAYLQMNSLRAEDTAVYYCSRWGGDGFYAMDYW
GQGTLVTVSSASTKGPSVFPLAPSSKSTSGGTAALGCLVKDYFPEPVTVSWNSGADD
DDDDGVHTFPAVLQSSGLYSLSSVVTVPSSSLGTQTYICNVNHKPSNTKVDKKVEPK
SCDKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLS
PGVEPKSCDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVS
NKALPAPIEKTISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHEALHNHYTQ
KSLSLSPGDDDDDD

The present antibody may be conjugated to an imaging or diagnostic agent.

A “therapeutic agent” as used herein refers to any agent that can be administered to a subject for the purpose of obtaining a therapeutic benefit of a disease or health-related condition. For example, antibodies conjugated to a therapeutic agent may be administered to a subject for the purpose of reducing the size of a tumor, reducing or inhibiting local invasiveness of a tumor, or reducing the risk of development of metastases.

A “diagnostic agent” or “imaging agent” (referred to interchangeably) as used herein refers to any agent that can be administered to a subject for the purpose of diagnosing a disease or health-related condition in a subject. Diagnosis may involve determining whether a disease is present, whether a disease has progressed, or any change in disease state.

The therapeutic or diagnostic agent may be a small molecule, a peptide, a protein, a polypeptide, an antibody, an antibody fragment, a DNA, or an RNA.

### A. Formulation and Administration

The present disclosure provides pharmaceutical compositions comprising bone-targeting antibodies. Such compositions comprise a prophylactically or therapeutically effective amount of an antibody or a fragment thereof, or a peptide immunogen, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, or delivered by mechanical ventilation.

Active vaccines are also envisioned where antibodies like those disclosed are produced in vivo in a subject at risk of Poxvirus infection. Such vaccines can be formulated for parenteral administration, e.g., formulated for injection via the intradermal, intravenous, intramuscular, subcutaneous, or even intraperitoneal routes. Administration by intradermal and intramuscular routes are contemplated. The vaccine could alternatively be administered by a topical route directly to the mucosa, for example by nasal drops, inhalation, or by nebulizer. Pharmaceutically acceptable salts, include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Passive transfer of antibodies, known as artificially acquired passive immunity, generally will involve the use of intravenous or intramuscular injections. The forms of antibody can be human or animal blood plasma or serum, as pooled human immunoglobulin for intravenous (IVIG) or intramuscular (IG) use, as high-titer human IVIG or IG from immunized or from donors recovering from disease, and as monoclonal antibodies (MAb). Such immunity generally lasts for only a short period of time, and there is also a potential risk for hypersensitivity reactions, and serum sickness, especially from gamma globulin of non-human origin. However, passive immunity provides immediate protection. The antibodies will be formulated in a carrier suitable for injection, i.e., sterile and syringeable.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### B. Hyperproliferative Diseases

While hyperproliferative diseases can be associated with any disease which causes a cell to begin to reproduce uncontrollably, the prototypical example is cancer. One of the key elements of cancer is that the cell's normal apoptotic cycle is interrupted and thus agents that interrupt the growth of the cells are important as therapeutic agents for treating these diseases. In this disclosure, a bone-targeting antibody may be used to treat a variety of types of cancers, such as bone cancers and cancers that metastasize to the bone.

Cancer cells that may be treated with the compounds of the present disclosure include but are not limited to cells from the bladder, blood, bone, bone marrow, brain, breast, colon, esophagus, gastrointestine, gum, head, kidney, liver, lung, nasopharynx, neck, ovary, prostate, skin, stomach, pancreas, testis, tongue, cervix, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In certain aspects, the tumor may comprise an osteosarcoma, angiosarcoma, rhabdosarcoma, leiomyosarcoma, Ewing sarcoma, glioblastoma, neuroblastoma, or leukemia.

### C. Methods of Treatment

In particular, the compositions that may be used in treating cancer in a subject (e.g., a human subject) are disclosed herein. The compositions described above are preferably administered to a mammal (e.g., rodent, human, non-human primates, canine, bovine, ovine, equine, feline, etc.) in an effective amount, that is, an amount capable of producing a desirable result in a treated subject (e.g., causing apoptosis of cancerous cells or killing bacterial cells). Toxicity and therapeutic efficacy of the compositions utilized in methods of the disclosure can be determined by standard pharmaceutical procedures. As is well known in the medical and veterinary arts, dosage for any one animal depends on many factors, including the subject's size, body surface area, body weight, age, the particular composition to be administered, time and route of administration, general health, the clinical symptoms of the infection or cancer and other drugs being administered concurrently. A composition as described herein is typically administered at a dosage that induces death of cancerous cells (e.g., induces apoptosis of a cancer cell), as assayed by identifying a reduction in cancer cell growth or proliferation.

The therapeutic methods of the disclosure (which include prophylactic treatment) in general include administration of a therapeutically effective amount of the compositions described herein to a subject in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, marker (as defined herein), family history, and the like).

In one embodiment, the disclosure provides a method of monitoring treatment progress. The method includes the step of determining a level of changes in hematological parameters and/or cancer stem cell (CSC) analysis with cell surface proteins as diagnostic markers (which can include, for example, but are not limited to CD34, CD38, CD90, and CD117) or diagnostic measurement (e.g., screen, assay) in a subject suffering from or susceptible to a disorder or symptoms thereof associated with cancer (e.g., leukemia) in which the subject has been administered a therapeutic amount of a composition as described herein. The level of marker determined in the method can be compared to known levels of marker either in healthy normal controls or in other afflicted patients to establish the subject's disease status. In preferred embodiments, a second level of marker in the subject is determined at a time point later than the determination of the first level, and the two levels are compared to monitor the course of disease or the efficacy of the therapy. In certain preferred embodiments, a pre-treatment level of marker in the subject is determined prior to beginning treatment according to the methods described herein; this pre-treatment level of marker can then be compared to the level of marker in the subject after the treatment commences, to determine the efficacy of the treatment.

### D. Additional Therapy

In certain embodiments, the compositions and methods of the present embodiments involve a bone-targeting antibody, in combination with a second or additional therapy. Such therapy can be applied in the treatment of any disease with bone tumors. For example, the disease may be a bone cancer or bone metastasis.

In certain embodiments, the compositions and methods of the present embodiments involve a bone-targeting antibody in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

The methods and compositions, including combination therapies, enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect, such as the killing of a cancer cell and/or the inhibition of cellular hyperproliferation. This process may involve contacting the cells with both an antibody or antibody fragment and a second therapy. A tissue, tumor, or cell can be contacted with one or more compositions or pharmacological formulation(s) comprising one or more of the agents (i.e., antibody or antibody fragment or an anti-cancer agent), or by contacting the tissue, tumor, and/or cell with two or more distinct compositions or formulations, wherein one composition provides 1) an antibody or antibody fragment, 2) an anti-cancer agent, or 3) both an antibody or antibody fragment and an anti-cancer agent. Also, it is contemplated that such a combination therapy can be used in conjunction with chemotherapy, radiotherapy, surgical therapy, or immunotherapy.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

An inhibitory antibody may be administered before, during, after, or in various combinations relative to an anti-cancer treatment. The administrations may be in intervals ranging from concurrently to minutes to days to weeks. In embodiments where the antibody or antibody fragment is provided to a patient separately from an anti-cancer agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations, it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

Various combinations may be employed. For the example below a bone-targeting antibody, is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

### 1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gamma1I and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein transferase inhibitors, transplatinum, and pharmaceutically acceptable salts, acids, or derivatives of any of the above.

### 2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

### 3. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen (Carter et al., 2008; Teicher 2014; Leal et al., 2014). Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach (Teicher 2009) and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints are molecules in the immune system that either turn up a signal (e.g., co-stimulatory molecules) or turn down a signal. Inhibitory checkpoint molecules that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present invention. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Application No. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVOR, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95 (17): 10067-10071; Camacho et al. (2004) J Clin Oncology 22 (145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WOO 1/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesions such as described in U.S. Pat. No. 8,329,867, incorporated herein by reference.

### 4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### 5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### III. Kits

In various aspects of the embodiments, a kit is envisioned containing therapeutic agents and/or other therapeutic and delivery agents. In some embodiments, the present embodiments contemplate a kit for preparing and/or administering an antibody composition of the embodiments. The kit may comprise one or more sealed vials containing any of the pharmaceutical compositions of the present embodiments. The kit may include, for example, engineered antibodies as well as reagents to prepare, formulate, and/or administer the components of the embodiments or perform one or more steps of the inventive methods. In some embodiments, the kit may also comprise a suitable container, which is a container that will not react with components of the kit, such as an eppendorf tube, an assay plate, a syringe, a bottle, or a tube. The container may be made from sterilizable materials such as plastic or glass.

The kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure of delivering a pharmaceutically effective amount of a therapeutic agent.

### IV. Examples

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Engineering Bone-Targeting Antibodies

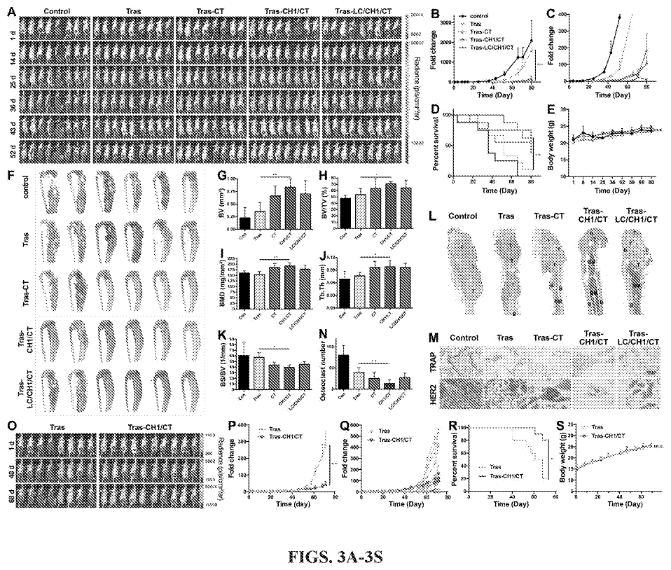

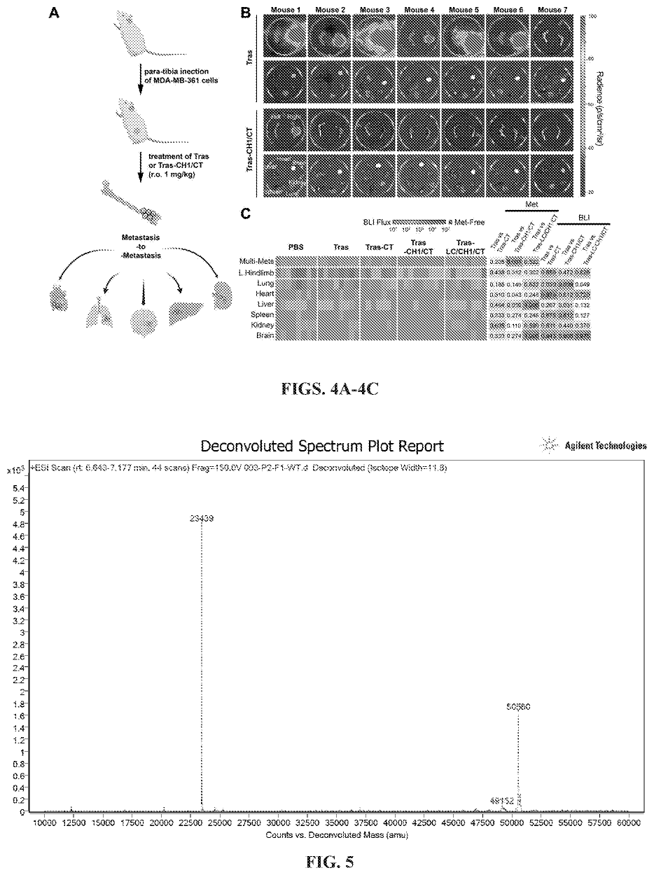

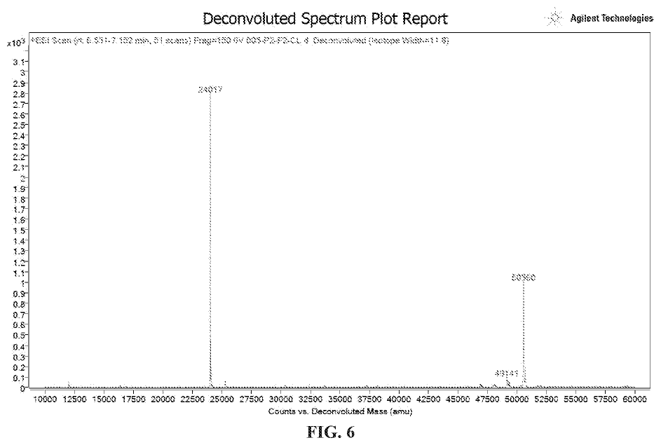

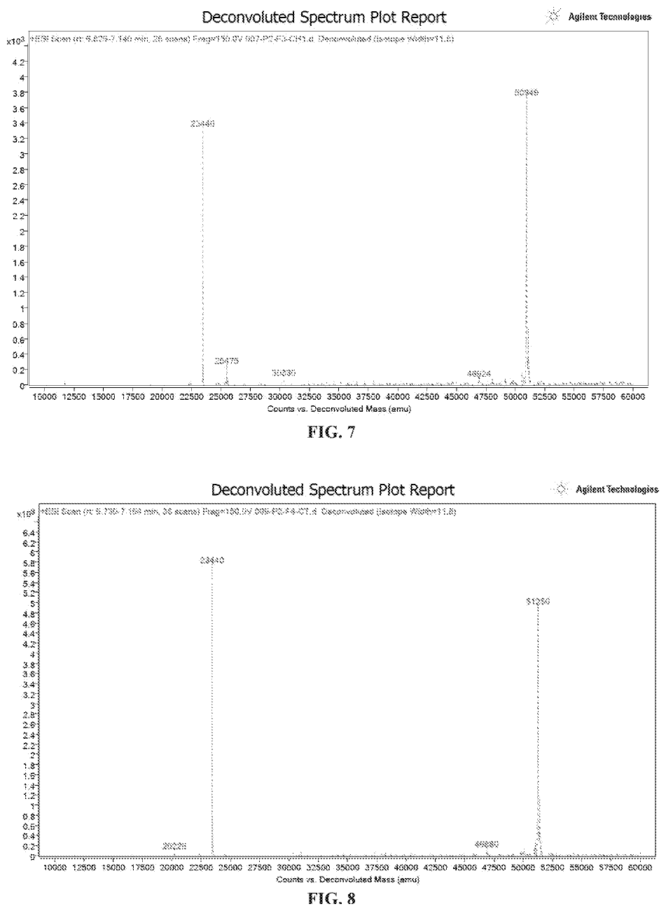

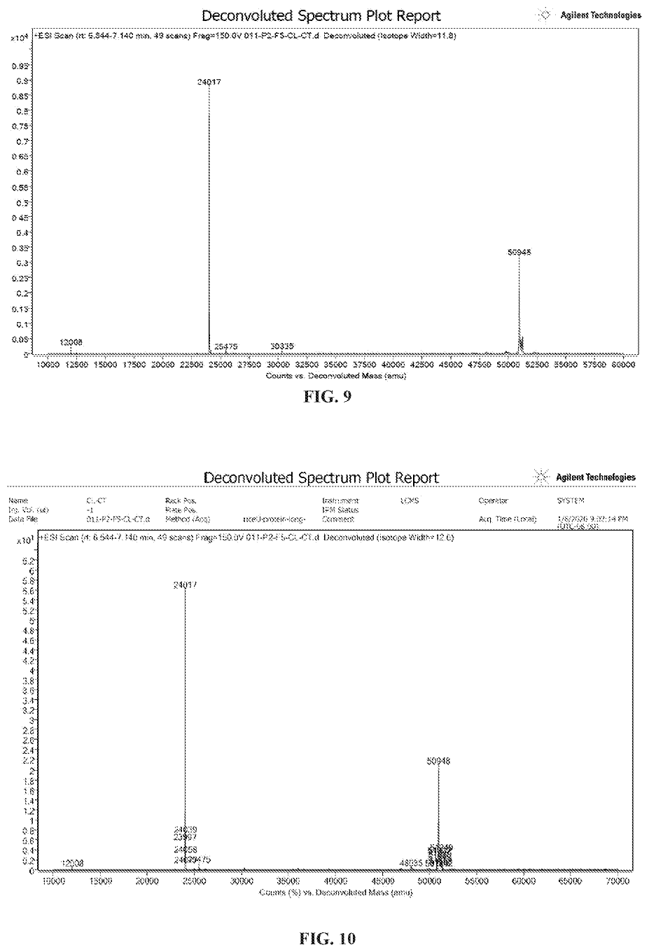

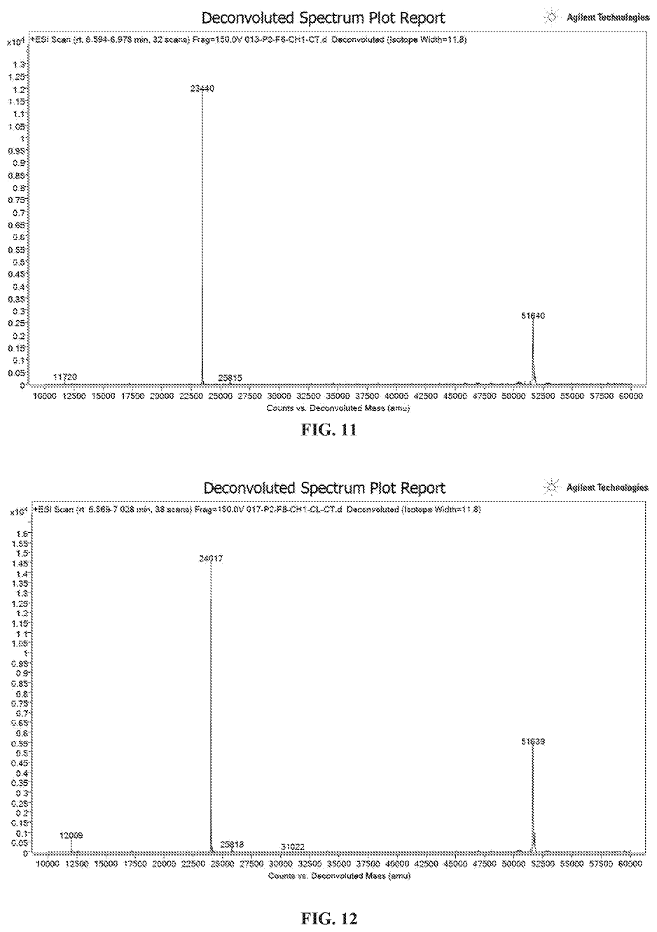

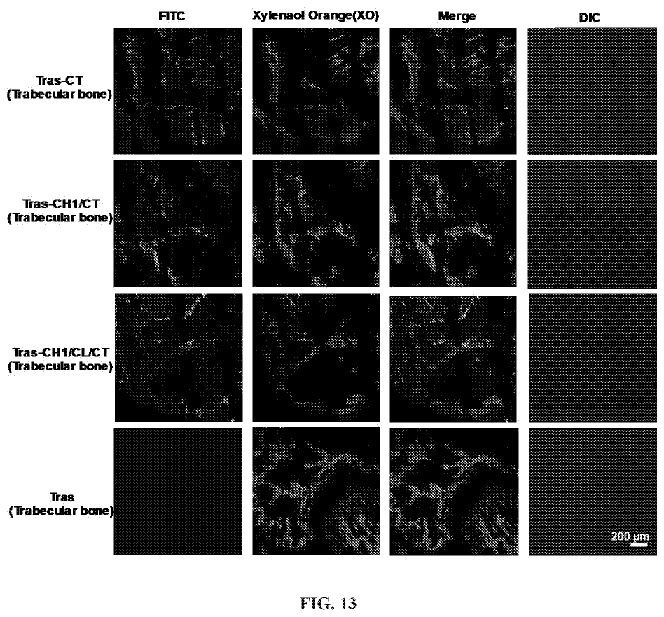

Modifying Trastuzumab with Bone-Homing Peptides. To harness the power of bone-homing peptide for selective delivery of antibodies to bone cancer sites, a library of antibodies was first engineered carrying the bone-homing peptide L-Asp₆ at various sites within the immunoglobulin molecules. The design principle was to install the bone-homing sequence at sites that would be minimally disruptive to the native IgG structure and function, yet would allow retention of the peptide's high affinity for bone matrix. Based on the crystal structure of an IgG1 monoclonal antibody, the L-Asp₆ peptide was inserted into permissive internal sites in the trastuzumab light chain (LC, A153), heavy chain (CH1, A165), and C-terminus (CT, G449) to yield Tras-LC, Tras-CH1, and Tras-CT, respectively (FIG. 2A). These internal sites have been shown to be stable to peptide insertion by screening an antibody peptide-placement library.²⁷ To modulate the bone tumor-targeting ability of engineered antibodies, the number of bone-homing peptide sequences were also varied per immunoglobulin molecule, generating trastuzumab species with two (Tras-LC/CT, Tras-CH1/CT, and Tras-LC/CH1) and three L-Asp₆ peptide sequences (Tras-LC/CH1/CT). The resulting seven constructs were expressed in ExpiCHO-S cells by transient transfection, followed by purification of immunoglobulins using protein G chromatography and analysis of expressed proteins by SDS-PAGE. All these antibody mutants were expressed in good yield (50-100 mg/L). SDS-PAGE and ESI-MS analysis confirmed the successful insertion of the bone-homing peptides (FIGS. 2B, 2C, 5-12). Among the antibody variants, the Tras-LC/CH1 species containing L-Asp₆ peptide sequences in both the light chain and heavy chain exhibited significant aggregation. Thus, this antibody mutant was not studied further.

In vitro Evaluation of Bone-Targeting Antibodies. With the bone-targeting antibody variants in hand, a HA binding assay was initially used to examine their binding to mineralized bone. Briefly, bone-targeting antibody species were incubated with HA for varying lengths of time, and unbound antibody remaining in solution was measured using a UV-Vis spectrophotometer. As shown in FIG. 2D, unmodified Tras exhibited only slight affinity for HA, while L-Asp₆ peptide-modified antibodies bound to HA in a time-dependent manner. Antibodies with multiple L-Asp₆ peptides, namely Tras-CH1/CT and Tras-LC/CH1/CT, exhibited the highest HA binding capacity, with over 80% of the antibody bound after 9 h of incubation (Table 2). Regarding the three antibody species containing single L-Asp₆ peptides, the C-terminal construct (Tras-CT) exhibited the highest HA binding capacity. Accordingly, fluorescein isothiocyanate (FITC)-labeled Tras, Tras-CT, Tras-CH1/CT, and Tras-LC/CH1/CT were used to stain non-decalcified bone sections from C57BL/6 mice. Sections treated with unmodified Tras exhibited no fluorescence after overnight incubation (FIG. 2E). In contrast, FITC signals were observed in all the sections stained with the three L-Asp₆ peptide-containing variants. These FITC antibody signals correlated well with the xylenol orange (XO) signal from the bone (FIG. 2E, 13).

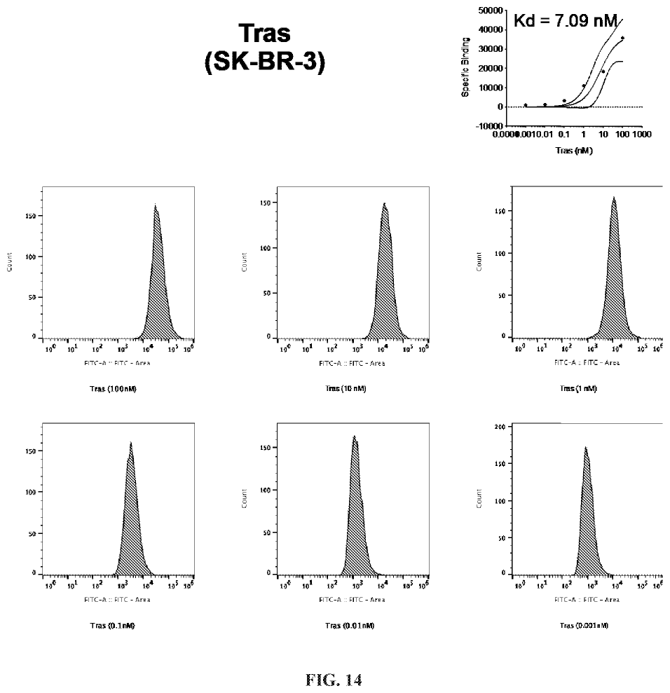

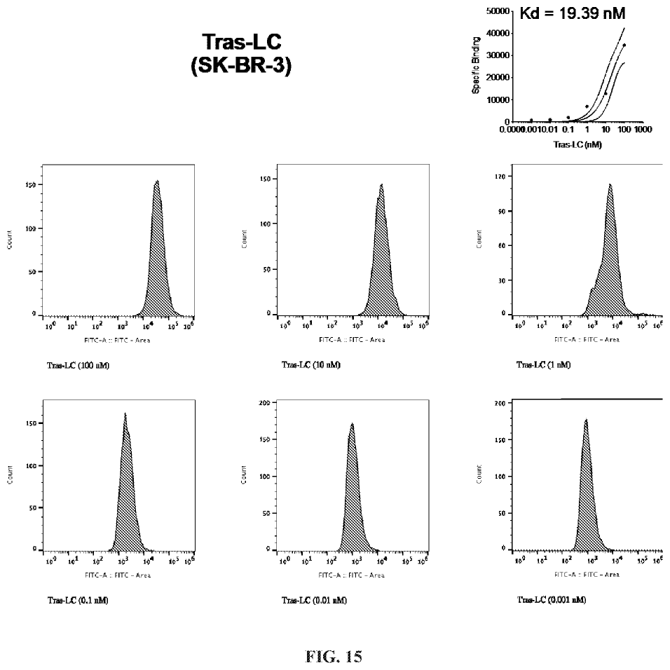

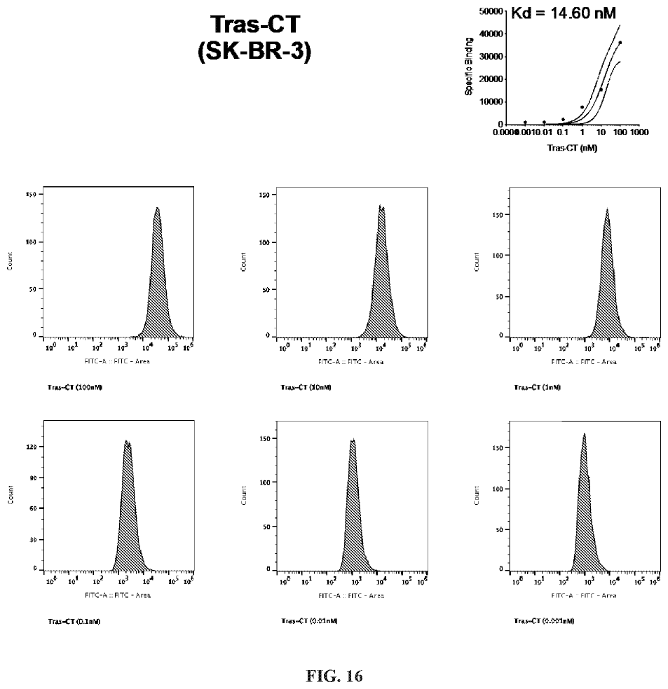

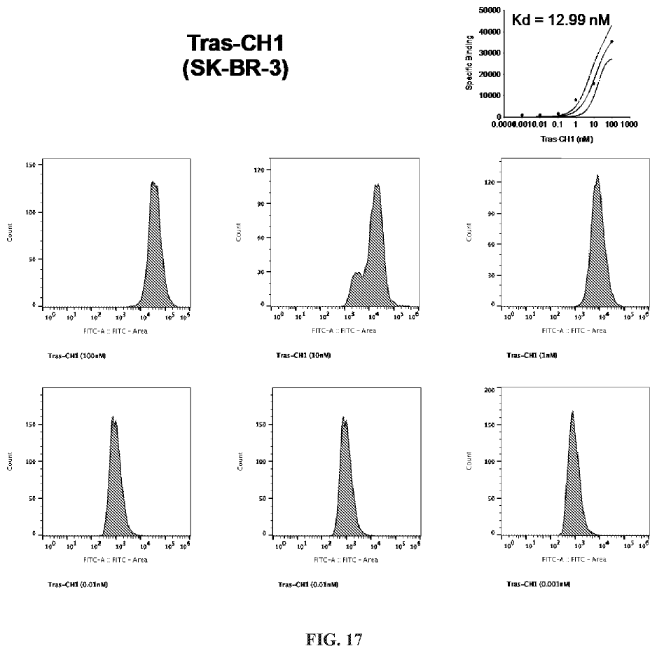

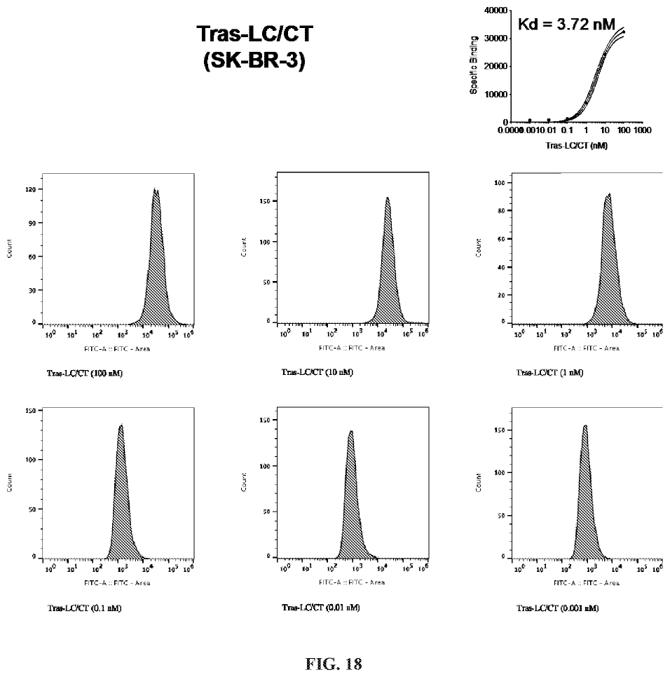

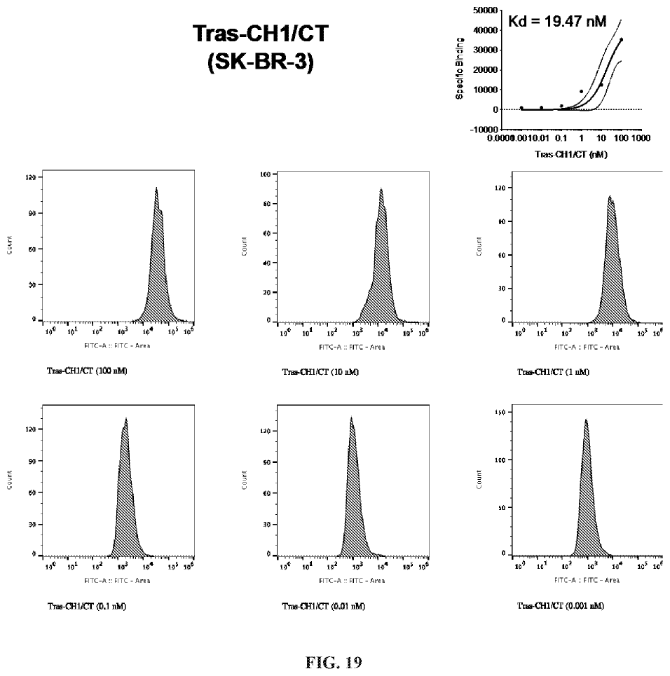

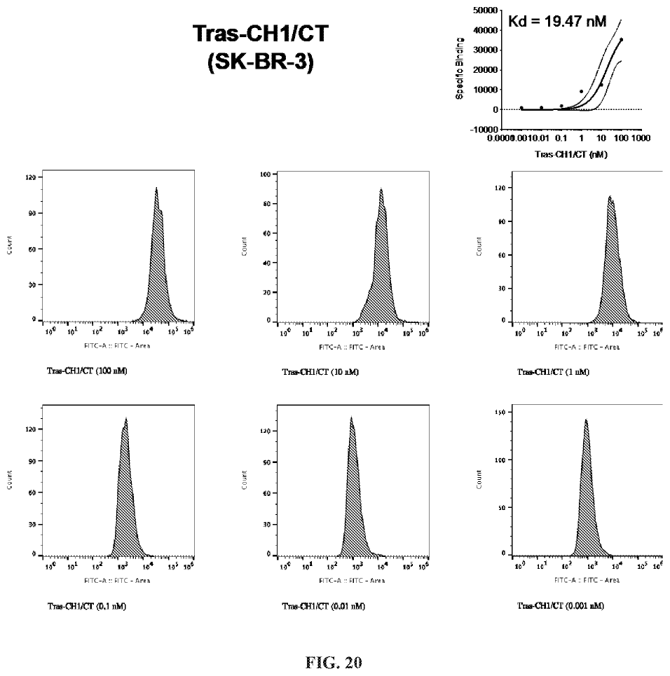

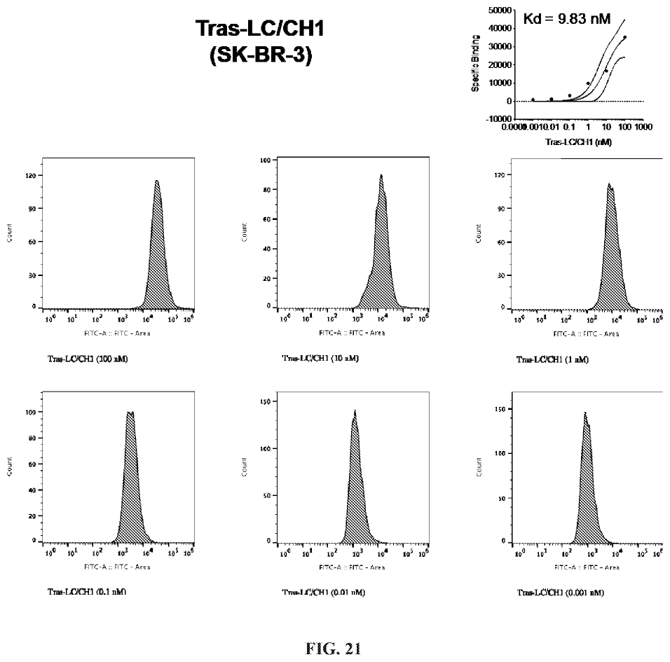

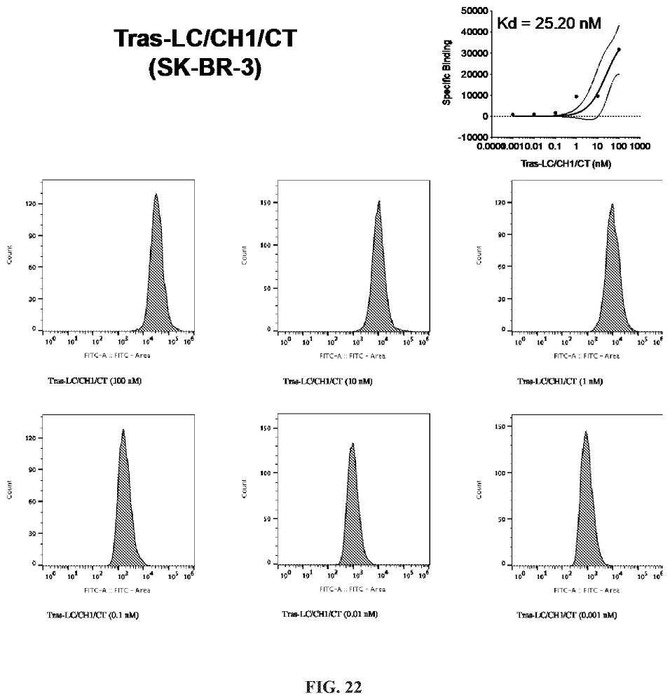

To demonstrate that insertion of the L-Asp₆ sequence had negligible influence on Tras antibody binding and specificity, FITC-labeled Tras, Tras-CT, Tras-CH1/CT, and Tras-LC/CH1/CT antibodies were tested for binding to HER2-positive and negative cell lines. Flow cytometry revealed that, while none of these antibodies bind to HER2-negative MDA-MB-468 cells, each of the bone-targeting antibodies bind to HER2-expressing SK-BR-3 cells with a K_{d} similar to that of unmodified Tras (7.09 nM) (FIGS. 2F, 14-23, and Table 3). The Tras-CT, Tras-LC, Tras-CH1, Tras-CH1/CT, Tras-CH1/CT and Tras-LC/CH1/CT species had slightly higher K_{d} values than Tras (14.60 nM, 19.39 nM, 12.99 nM, 19.47 nM, 19.47 nM and 25.20 nM, respectively) (FIGS. 14-22), likely due to increased electrostatic repulsion mediated by the inserted negatively charged residues. The in vitro cytotoxicity of the bone-targeting antibodies was next evaluated against HER2-positive and negative cell lines. Consistent with the flow cytometry data, Tras-CT, Tras-CH1/CT, and Tras-LC/CH1/CT antibodies kill SK-BR-3 cells with an efficiency similar to that of unmodified Tras (EC₅₀ values of 5.97±5.64 nM, 13.07±12.09 nM, and 21.10±20.25 nM, respectively (FIG. 2G). In contrast, none of the antibodies exhibit cytotoxicity against HER2-negative MDA-MB-468 cells under the same experimental conditions (FIG. 2G). These results indicate that introduction of the bone-homing sequence into antibodies significantly enhanced their bone affinity while preserving their anti-tumor activities. To explore the potential toxicity of Tras-CH1/CT on bone cells, murine preosteoclast cell line RAW264.7 and osteoblast cell line MC3T3-E1 were incubated with various concentrations of Tras or Tras-CH1/CT for 4 days, and cell survival was assessed. As shown in the FIG. 38, neither Tras nor Tras-CH1/CT showed significant toxicity. These results indicate that, despite the enhanced binding to the bone, bone-targeting antibodies are unlikely to cause significant toxicity toward bone stromal cells.

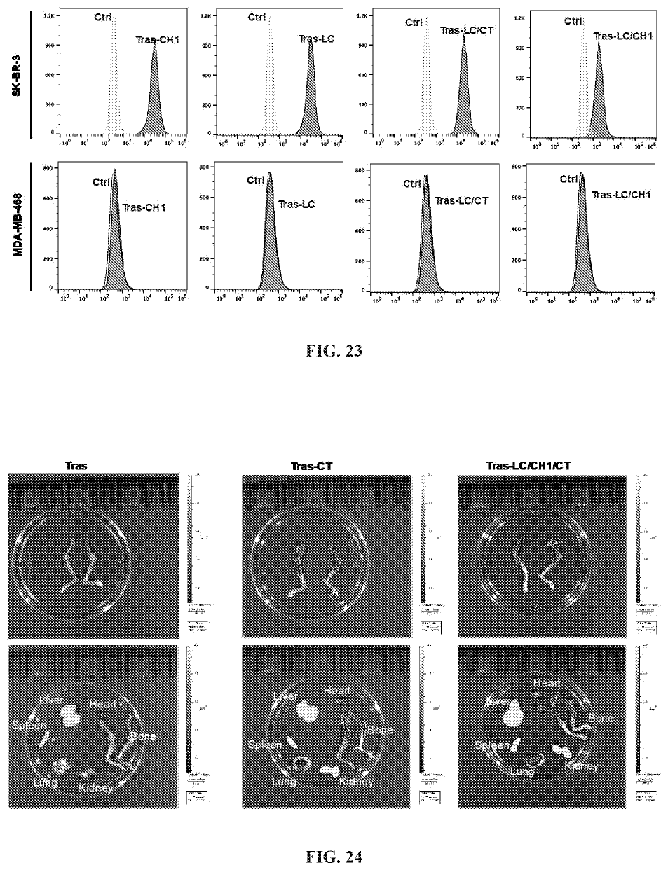

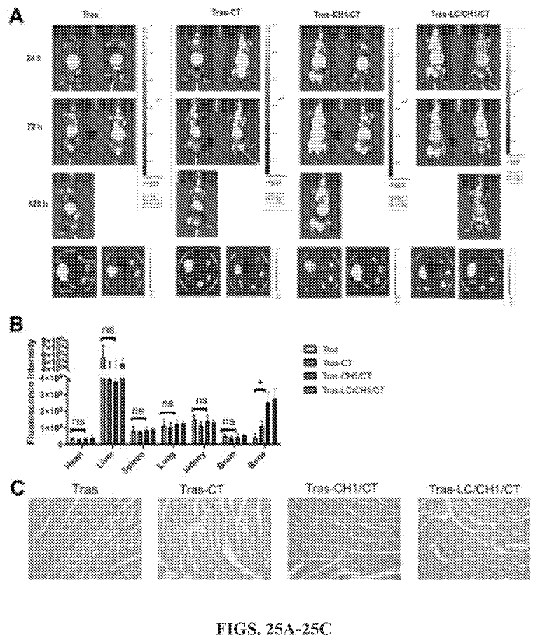

In vivo Distribution of Bone-Targeting Antibodies. The effects of bone-homing peptides on Tras antibody distribution were further investigated in a mouse xenograft model. Using para-tibial injection, 2×10⁵ HER2-expressing MDA-MB-361 breast cancer cells labeled with firefly luciferase and red fluorescent protein were first introduced into the right leg of nude mice, followed by administration of sulfo-Cy7.5 labeled Tras, Tras-CT, Tras-CH1/CT, or Tras-LC/CH1/CT via retro-orbital injection. 72 h and 120 h after antibody infusion, the major organs were collected and imaged for antibody distribution. The intensity of the interosseous fluorescence signal was stronger in the Tras-CT, Tras-CH1/CT, and Tras-LC/CH1/CT injected animals than in Tras injected mice (FIGS. 2H, 24, and 25). As shown in FIG. 25, the quantified ex vivo signals from different tissues suggest that a significantly higher signal of bone-targeting antibody could be observed in the skeletal tissues, but not in other tissues (FIG. 25B). Furthermore, the inventor performed a histological analysis of collected heart tissues and did not observe obvious pathological variation in cardiac tissues upon different treatments, indicating good biocompatibility of bone-targeting antibodies (FIG. 25C). Moreover, larger quantities of bone-targeting antibodies were present in tumor-bearing right leg bones compared to left healthy bone, likely due to L-Asp₆ peptide-mediated targeting to the bone resorption niche. Overall, these results indicated that introduction of the L-Asp₆ sequence can significantly increase the concentration of therapeutic antibody in bone tumor sites. This effect has the potential to enhance antitumor activity at the tumor site, while at the same time decreasing systemic toxicity.

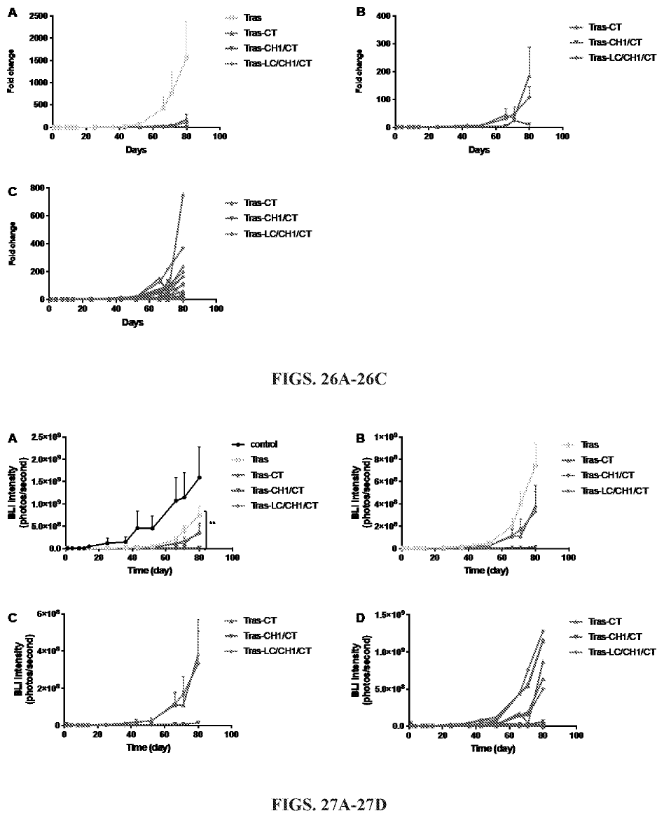

In vivo Therapeutic Activity of Bone-Targeting Antibodies against Bone Micrometastases. To determine whether bone-targeting antibodies can serve as novel therapeutic entities for the treatment of breast cancer metastasis to bone, in vivo antitumor experiments were performed in nude mice bearing MDA-MB-361 tumors. 2×10⁵ MDA-MB-361 breast cancer cells labeled with firefly luciferase and red fluorescent protein were inoculated into the right leg of nude mice via para-tibial injection. One week after injection, wild type Tras and bone-targeting Tras antibodies were administered by retro-orbital injection. As shown in FIG. 3A, mice receiving 1 mg/kg of unmodified Tras did not respond well to this treatment. Despite an initial inhibitory effect during the first two weeks of treatment, unmodified Tras failed to control long-term tumor growth, prolonging median mouse survival of subjects by only 9.7 days (FIGS. 3B and 3C). In contrast, when the tumor-bearing mice were treated with bone-targeting antibodies, tumor growth was significantly inhibited. Tras-CT-, Tras-CH1/CT-, and Tras-LC/CH1/CT-treated groups exhibited pronounced delays in tumor growth of 27.5, 35.8 and 18.9 days, respectively (FIGS. 3B and 3C). FIGS. 3A, 3B, 3C, 26, 27, and Table 4 show the bioluminescent (BLI) signals for each treatment groups from day 1 to day 80. In the PBS-treated control group, there was a progressive increase in the BLI signal over time. The BLI signals from day 1 to 80 demonstrated that bone-targeting Tras antibody-treated groups experienced significant delays in tumor growth compared to the Tras-treated group (FIGS. 3B and 3C). Mice treated with Tras-CH1/CT exhibited the smallest increases in tumor size (Tras-CH1/CT vs Tras: 9.3±4.2 vs 1562.7±801.6, p<0.0001). Furthermore, the Tras-CH1/CT-treated mice experienced a 62.5% rate of survival, a significant improvement over that seen in Tras-treated mice (FIG. 3D). Thus, treatment with Tras-CH1/CT appears to resulted in more effective inhibition of micrometastasis progression than that seen in Tras-treated mice. Treatment with bone-targeting antibodies was well tolerated, with no overt signs of toxicity observed in any of the treatment groups. For example, no differences in body weights were observed across the various treatment groups (FIG. 3E).

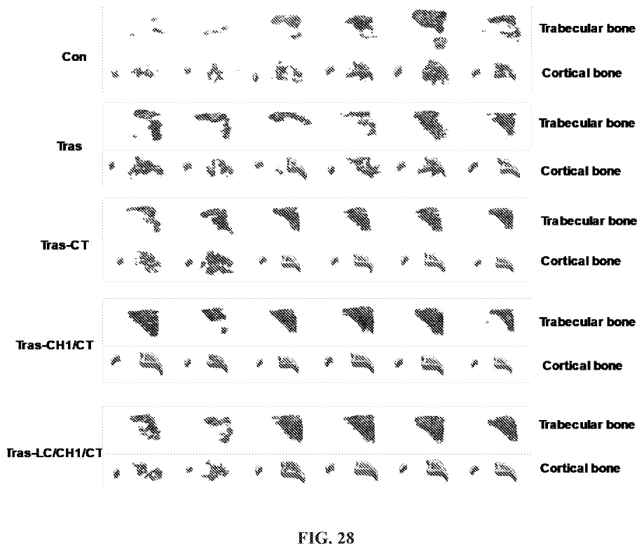

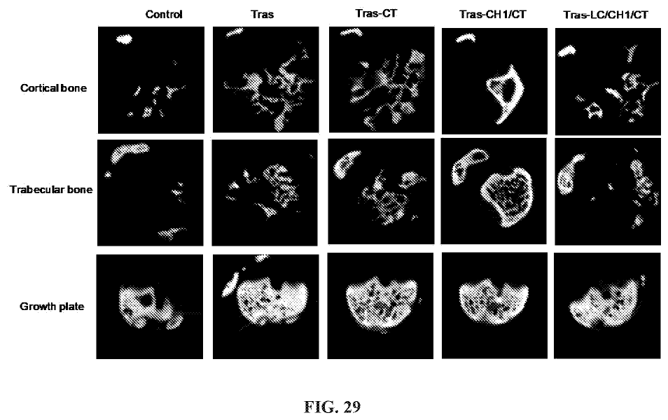

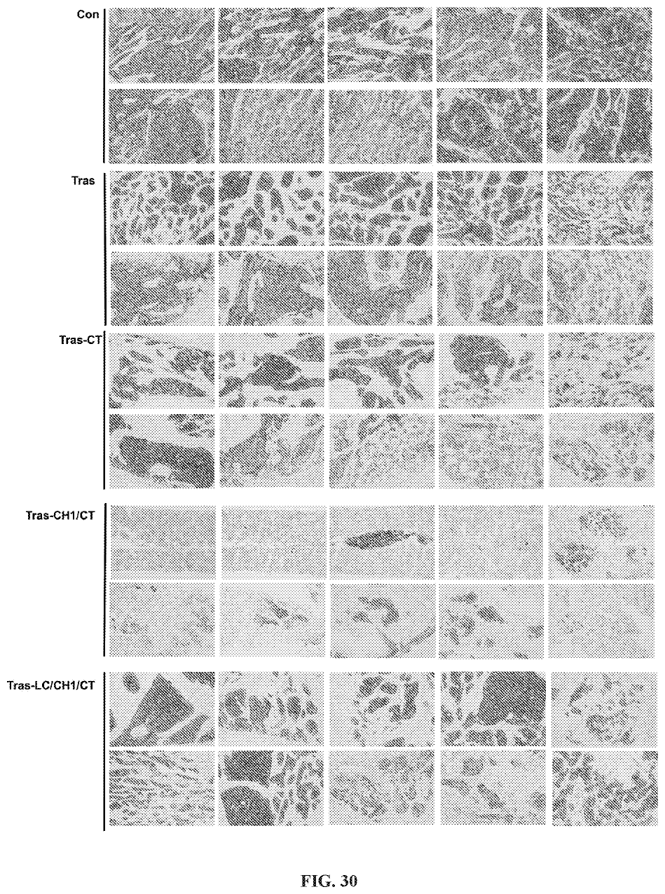

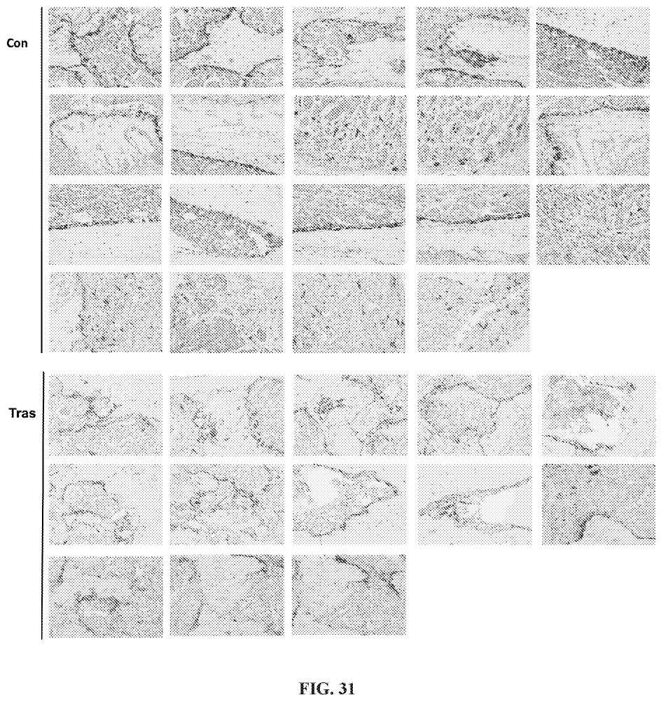

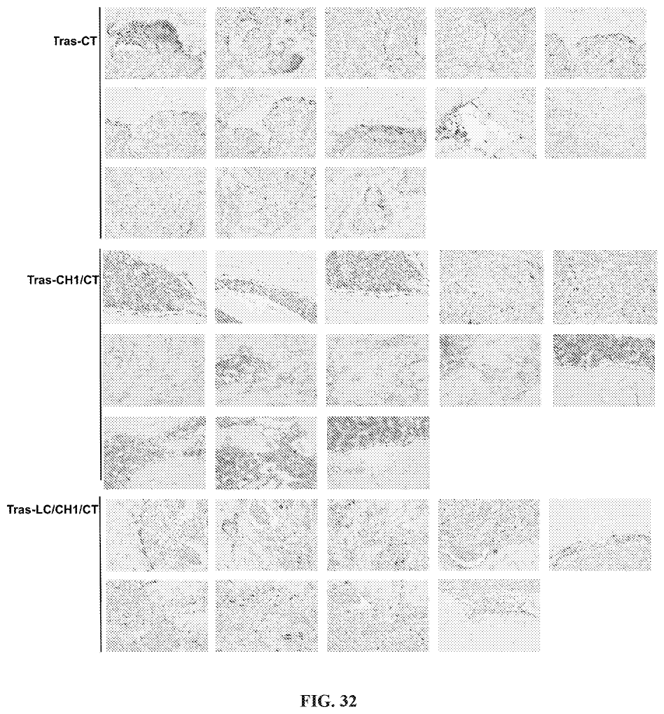

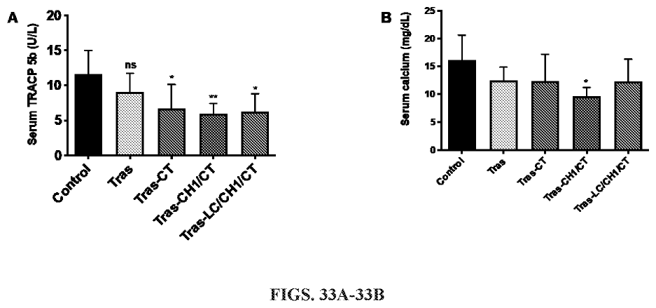

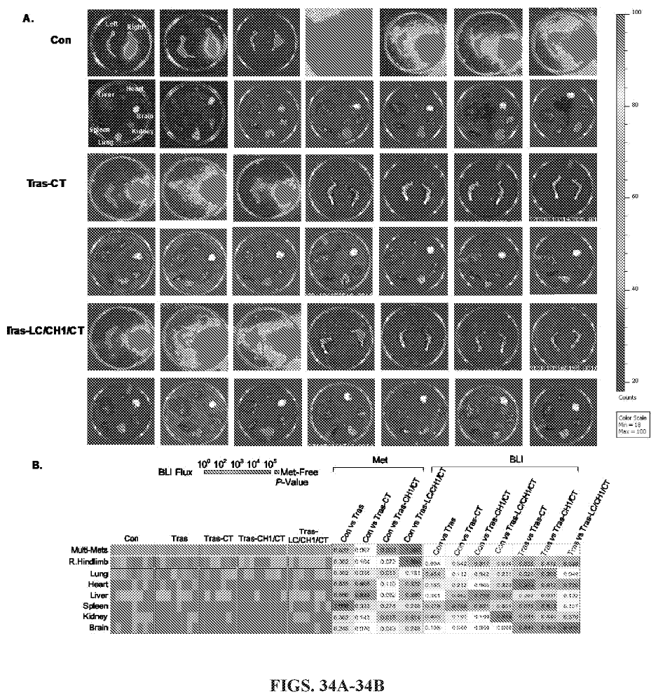

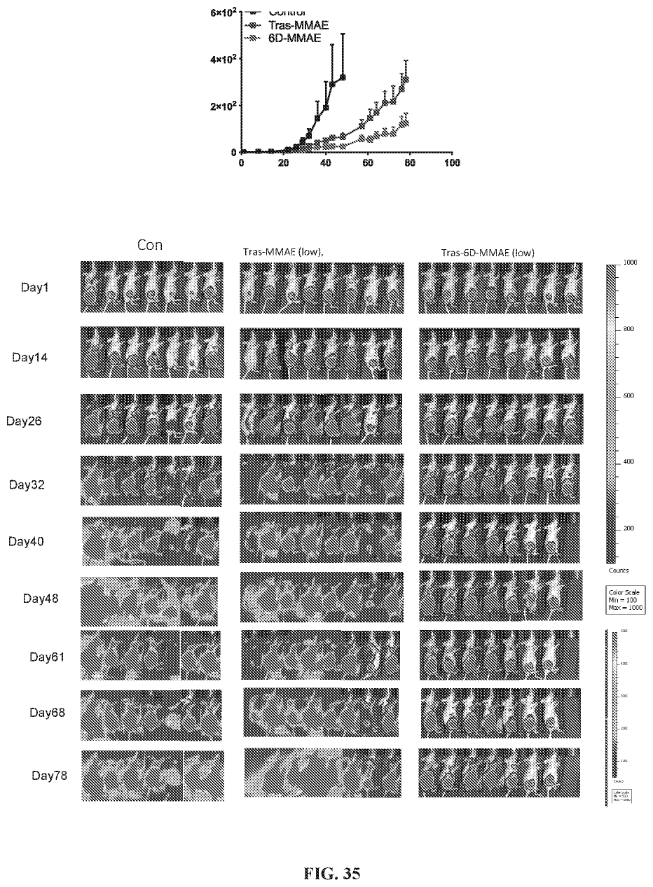

At the end of the experiment (day 81), tibiae (from tumor-bearing legs) were harvested and scanned by micro-computed tomography (micro-CT). The micro CT analysis revealed extensive osteolytic bone destructions in both the PBS- and Tras-treated groups, while bone loss was significantly reduced in bone-targeting antibody-treated groups (FIGS. 3F, 28 and 29). Compared to mice in the PBS- and Tras-treated groups, Tras-CH1/CT-treated mice exhibited greater bone volume (BV, FIG. 3G), greater bone volume/tissue volume ratio (BV/TV, FIG. 3H), greater bone mineral density (BMD, FIG. 3I), and thicker trabecular bone (Tb.Th, FIG. 3J), but smaller bone surface/bone volume ratio (BS/BV, FIG. 3K and Table 5). These parameters are all indicative of significant retardation of micrometastasis-induced osteolysis by antibodies modified with bone-homing peptides. Histological analysis further revealed the invasion of tumor cells into the bone matrix and into the adjacent tissue in the Tras-treated group (FIG. 3L). Histology also confirmed the reduction of intratibia tumor burden in these mice that was indicated by the BLI results. Bone sections from Tras-CH1/CT treated mice revealed the reduced tumor growth and relatively normal bone morphology in these mice, consistent with significant inhibition of tumor invasion and bone destruction (FIG. 3L). Bone samples from the various treatment groups were also analyzed for bone resorbing TRAP (tartrateresistant acid phosphatase) positive multinucleated osteoclasts (shown as pink cells) and HER-expressing cancer cells (FIGS. 3M, 3N, 30-32). Compared to Tras treatment, Tras-CH1/CT treatment significantly reduced the numbers of both osteoclasts and HER2-positive cells in bone niches, once again consistent with the ability of bone targeting antibodies to inhibit micrometastasis progression (FIG. 3N). Given that tumor-induced hypercalcemia and TRACP 5b protein are the indicators of osteolytic bone destruction, the effects of bone-targeting antibody-treated were evaluated. The results suggested that Tras-CH1/CT-treated groups had better therapeutic effects (FIG. 33).

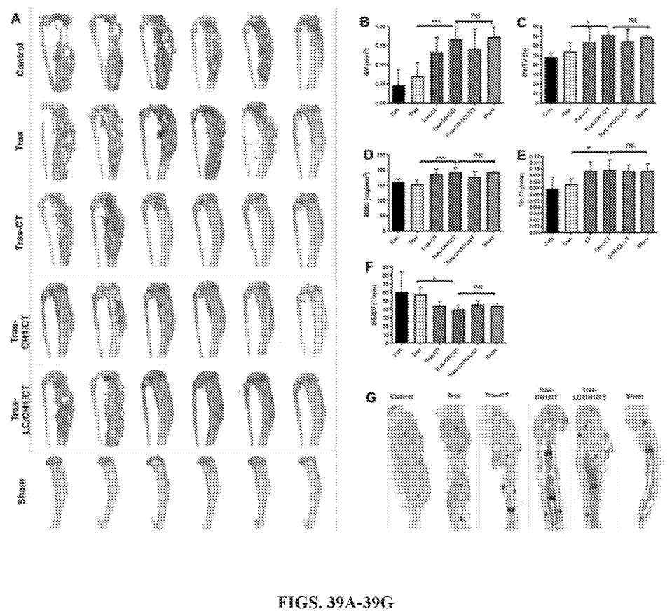

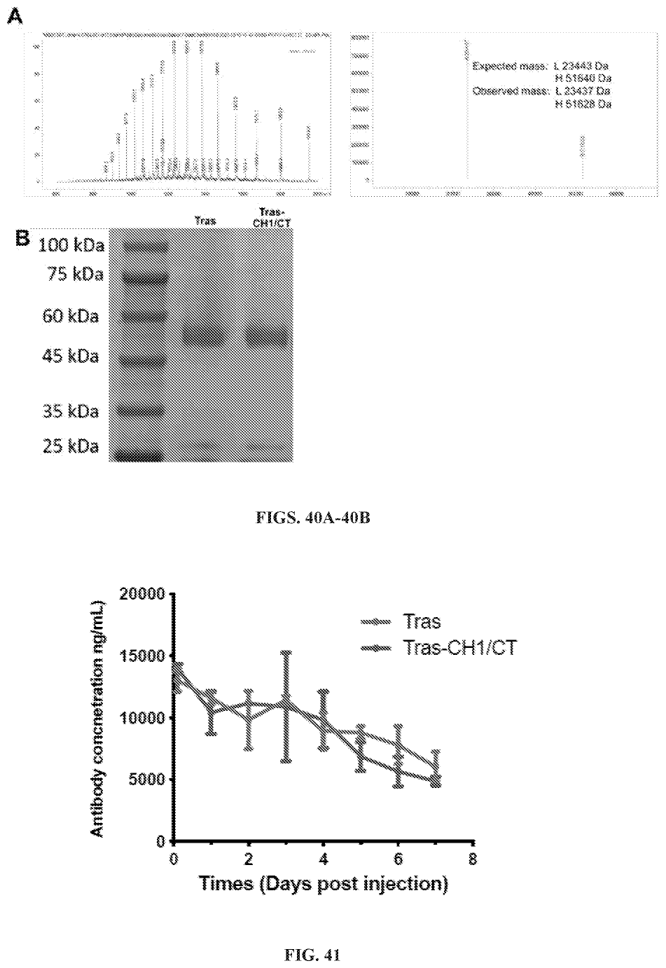

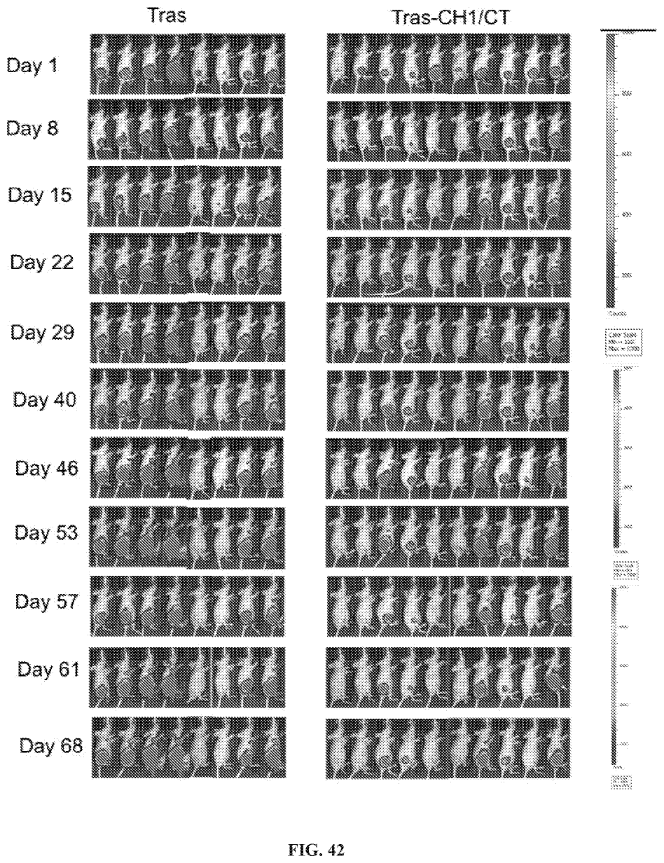

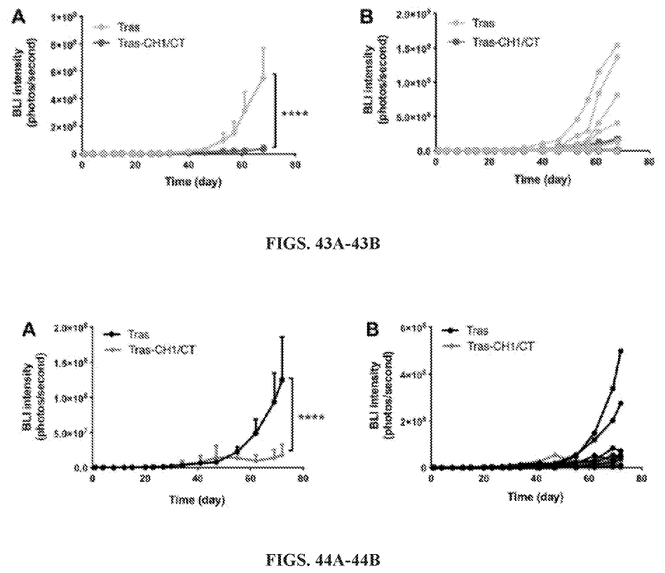

Consistent with the microCT and histological analysis, Tras-CH1/CT treatment showed the lowest level of bone destruction as evaluated by hypercalcemia and TRACP 5b protein levels in the serum (FIG. 33). To evaluate the stability of the bone-targeting antibodies in vitro, Tras and Tras-CH1/CT (1 mg/mL, 100 μL) were incubated in PBS at 4° C. for 3 months. SDS-PAGE and ESI-MS analysis revealed that no significant degradation or aggregation of bone-targeting antibodies was observed (FIG. 40). In the meantime, the serum levels of both Tras and Tras-CH1/CT showed a similar pharmacokinetics in vivo (FIG. 41), suggesting that the addition of bone-homing peptides does not alter the stability of antibodies. Next, the benefits of bone-targeting antibodies for bone metastasis were evaluated at a higher dose. Nude mice with MDA-MB-361 bone metastases were treated with Tras or Tras-CH1/CT at 10 mg/kg every 2 weeks. Notably, Tras-CH1/CT treatment results in statistically significant growth inhibition and prolonged overall survival, compared to treatment with 10 mg/kg of Tras (FIGS. 3O-R, 42, 43). Furthermore, no weight loss was observed with a high dose of bone-targeting antibodies (FIG. 3S).

The enhanced therapeutic efficacy of bone-targeting antibodies was also evaluated with a secondary bone metastasis model using MCF-7 breast cancer cells. Consistent with MDA-MB-361 models, a significant reduction in metastatic burden and increase of mouse survival were observed in the Tras-CH1/CT-treated group, compared to the Tras-treated group (FIGS. 36A-D, 44). Tras-CH1/CT treatment did not alter additional weight change in animals (FIG. 36E).

To examine the efficacy of Tras-CH1/CT in treating primary tumors, MDA-MB-361 cells (1×10⁶) were injected in mammary fat pads followed by the treatment of PBS, Tras (1 mg/kg), or Tras-CH1/CT (1 mg/kg). As shown in FIGS. 45A-C, while both Tras and Tras-CH1/CT treatments significantly decreased the tumor growth in the mammary fat pads, there were no statistical differences in tumor size between two treatments (FIG. 45C). Taken together, these data suggest that the introduction of bone-homing peptides into antibodies can significantly inhibit breast cancer bone metastases without compromising its antitumor activity in primary tumors.

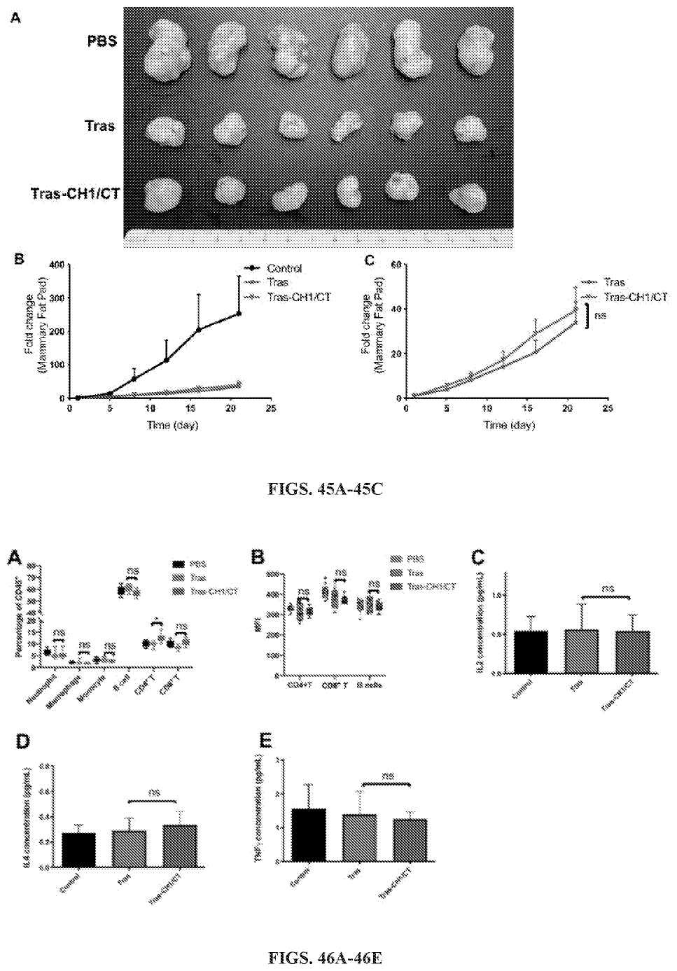

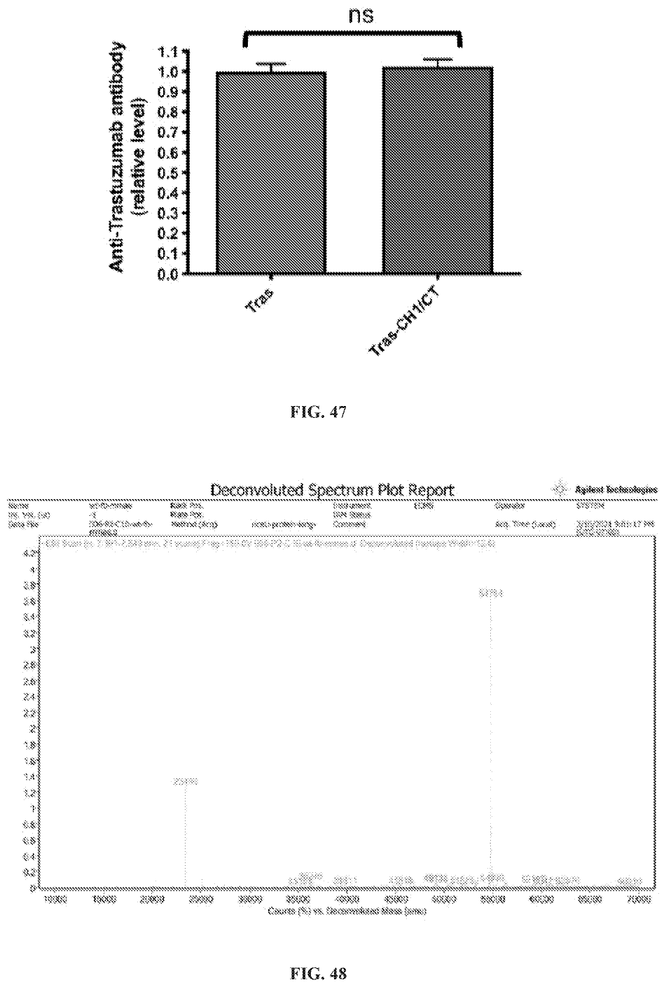

Immunogenicity Assessment of Bone-Targeting Antibodies. To understand if the treatment of bone-targeting antibodies causes specific immune responses, immunocompetent C57BL/6J mice were treated with PBS, Tras, or Tras-CH1/CT twice a week for 2 weeks. As shown in Figure S37A, the immunoprofiling determined by flow cytometry suggests that mice treated with either Tras or Tras-CH1/CT had similar immune composition except CD4+ T cells. Furthermore, the IFNγ staining of CD4+ T cells had no significant difference in Tras- and Tras-CH1/CT-treated mice, indicating that Tras-CH1/CT does not significantly alter the functional activity of CD4+ T cells (FIG. 46B). Furthermore, there were no significant differences in serum levels of INFγ, IL-2, and IL-4 levels between treatments with wild-type antibodies or bone-targeting antibodies (FIGS. 46C-E). The results indicated that adding L-Asp₆ to antibodies does not cause any obvious extra immune response. Furthermore, the in vivo immune response to the bone-targeting antibodies can also be tested by an immunogenicity test based on an anti-trastuzumab antibody ELISA-based assay as previously reported. Similar anti-trastuzumab antibody levels were observed in animals treated with Tras (5 mg/kg) or Tras-CH1/CT (5 mg/kg), which suggests that the addition of bone targeting peptides will not alter the immunogenicity of antibodies (FIG. 47).

Bone-Targeting Antibodies Inhibit Secondary Metastases from Bone Lesions. In more than two-thirds of patients, breast cancer metastases are not restricted to the skeleton, but subsequently also occur in other organs.^{28,29,30,31} Recent genomic analyses suggest that these metastases, the major cause of morbidity and mortality, are not derived from primary tumors, but are seeded from other metastatic sites. Taking advantage of a recently developed approach that selectively delivers cancer cells to hind limb bones, frequent “metastasis-to-metastasis” seeding from established bone lesions to multiple other organs has been observed.³²⁻³⁴ Hence, it was evaluated whether bone-targeting antibodies can inhibit these secondary metastases derived from bone lesions. Using the para-tibiae injection method, highly bone-specific tibiae-bearing tumors were at early stages of development. As the bone lesion progresses, metastases marked by bioluminescence signals began to appear in many other organs, including other bones, lungs, heart, liver, spleen, kidney, and brain. Using this model, 2×10⁵ luciferase-labeled MDA-MB-361 cells were introduced into the right hind limbs of nude mice via para-tibial injection, followed by treatment with unmodified Tras and bone-targeting Tras antibodies. Mice were euthanized 81 days after initiation of treatment, and organs were harvested for ex vivo bioluminescence imaging. Seven organs including the right hindlimb bone and six soft tissue organs were isolated for assessment of metastasis. As shown in FIGS. 4A, 4B and 34, treatment with Tras-CH1/CT significantly reduced the frequency of secondary metastasis to the contralateral hind limb (left hindlimb bone), heart, and liver. The ex vivo BLI intensities of secondary metastases from primary bone lesions are reduced in experimental groups treated with bone-targeting antibodies, especially in the Tras-CH1/CT-treated group. Compared to treatment with unmodified Tras, the metastatic signals from the lung and liver were significantly decreased after the Tras-CH1/CT treatment (FIG. 4B). Taken together, these data reveal the enhanced therapeutic efficacy of bone-targeting antibodies against both initial bone metastasis and secondary metastasis from the bone to other distant organs.

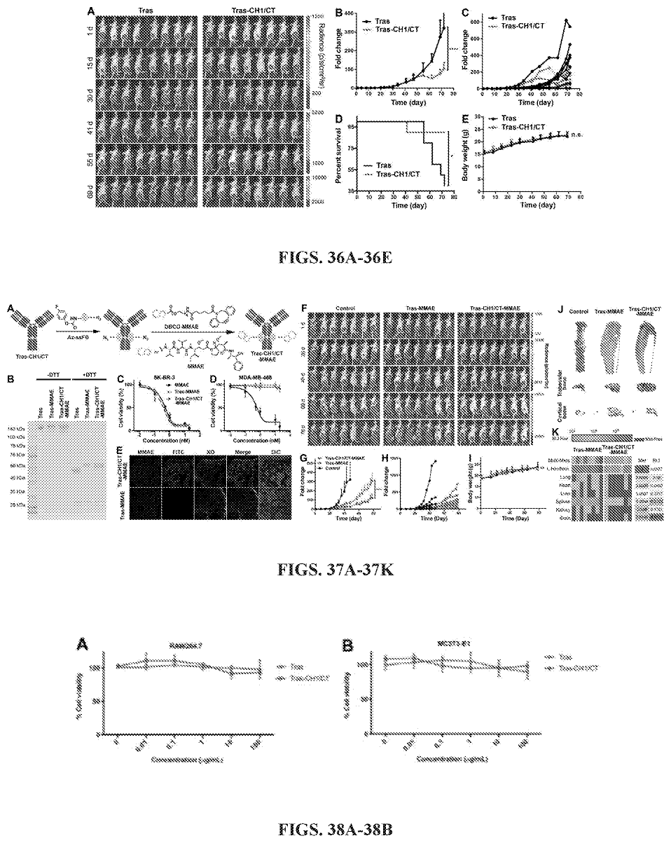

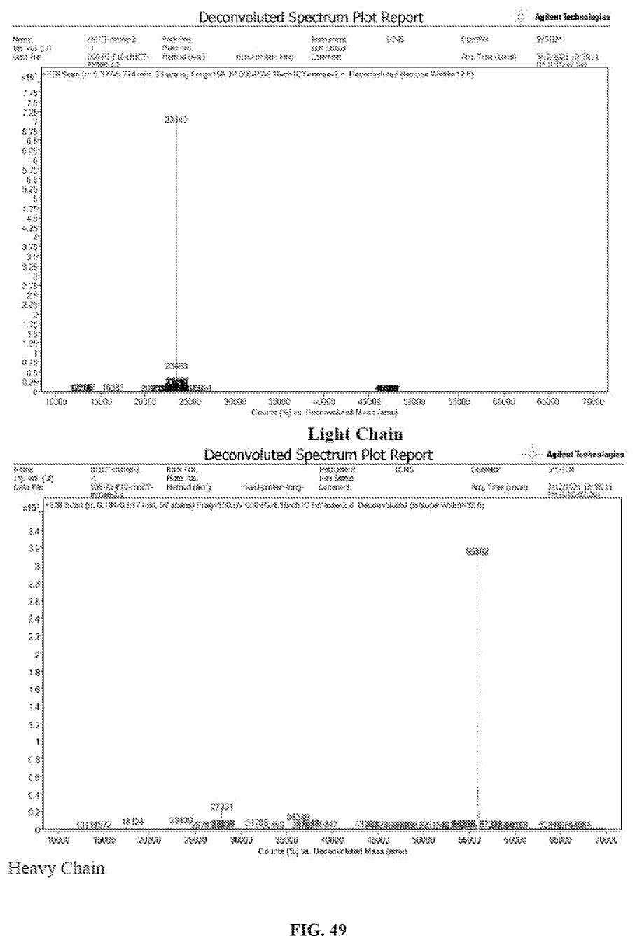

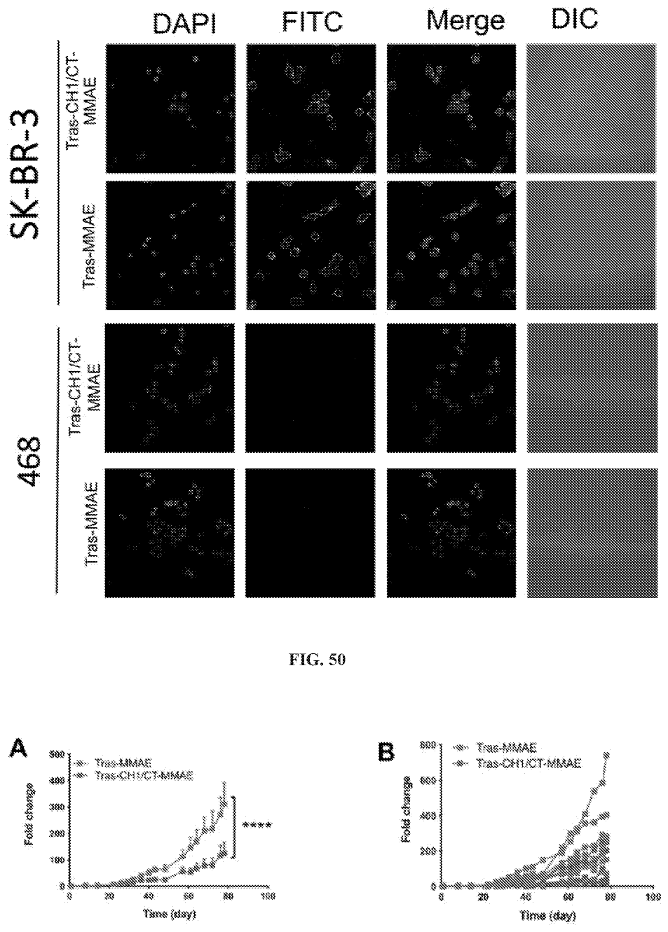

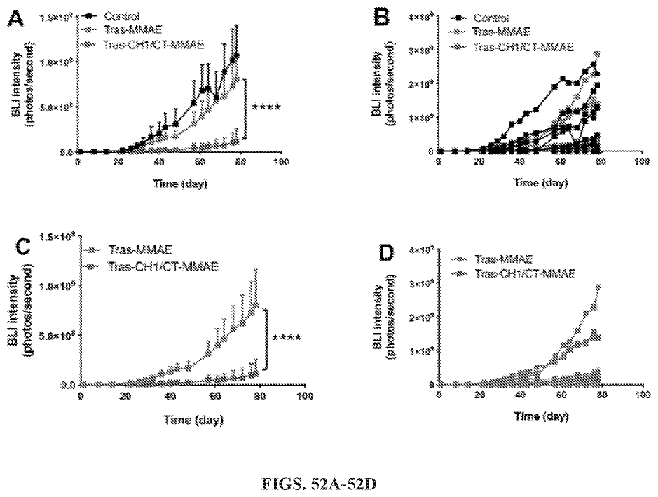

Modification of Antibody-Drug Conjugates with the Bone-Homing Peptide Exhibit Enhanced Therapeutic Efficacy in vivo. Antibody-drug conjugates (ADCs) that combine the antibody's tumor specificity with the high toxicity of chemotherapy drugs are emerging as an important class of anticancer drugs for breast cancer patients, especially ones with advanced breast cancer. Following the first FDA approval of trastuzumab emtansine (T-DM1) for HER2-positive breast cancer, trastuzumab deruxtecan has been recently approved for the treatment of adults with unresectable or metastatic HER2-positive breast cancers. To test if bone-targeting ADCs can further improve their efficacy in treating bone metastases, pClick conjugation technology was first used to site-specifically couple the monomethyl auristatin E (MMAE) to both wild-type antibody Tras and the bone-targeting antibody Tras-CH1/CT (FIG. 37E). The successful conjugation was demonstrated by SDS-PAGE and ESI-MS (FIGS. 37B, 48, 49). To ensure that conjugation of toxin did not alter the antigen targeting ability and specificity, the in vitro binding assays were performed using HER2-positive and -negative cells (FIG. 50). Tras-CH1/CT-MMAE showed a high binding affinity to HER2-positive SK-BR-3 cells, but not HER2-negative MDA-MB-468 cells. Next, the in vitro cytotoxicity of these ADCs was evaluated in SK-BR-3 and MDA-MB-468 breast cancer cell lines (FIGS. 37C-D). Both Tras-MMAE and Tras-CH1/CT-MMAE exhibited high potency only in the SK-BR-3 (EC50:0.18±0.82 nM and 0.49±0.30 nM, respectively), with no significant toxicity was observed in the MDA-MB-468 cells. To test the bone targeting ability of Tras-CH1/CT-MMAE in vitro, either Tras-CH1/CT-MMAE or Tras-MMAE were incubated with nondecalcified bone sections. As expected, only the signal of Tras-CH1/CT-MMAE correlated well with the XO signal, confirming its bone-targeting ability (FIG. 37E).

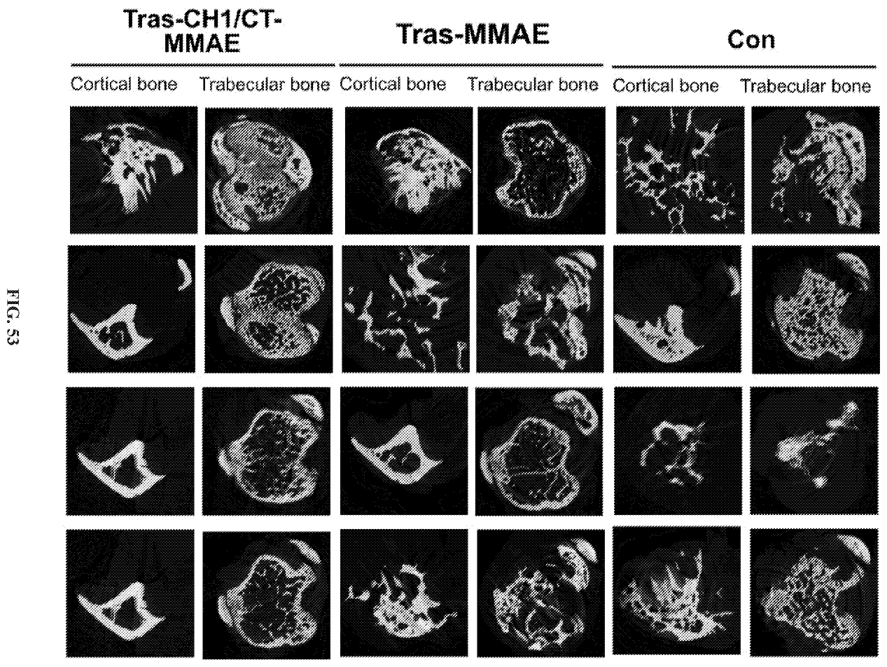

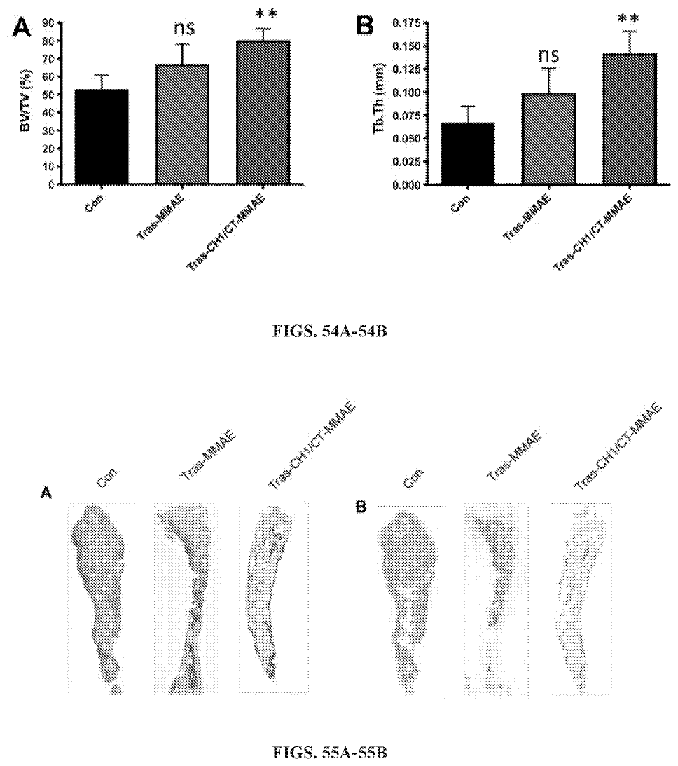

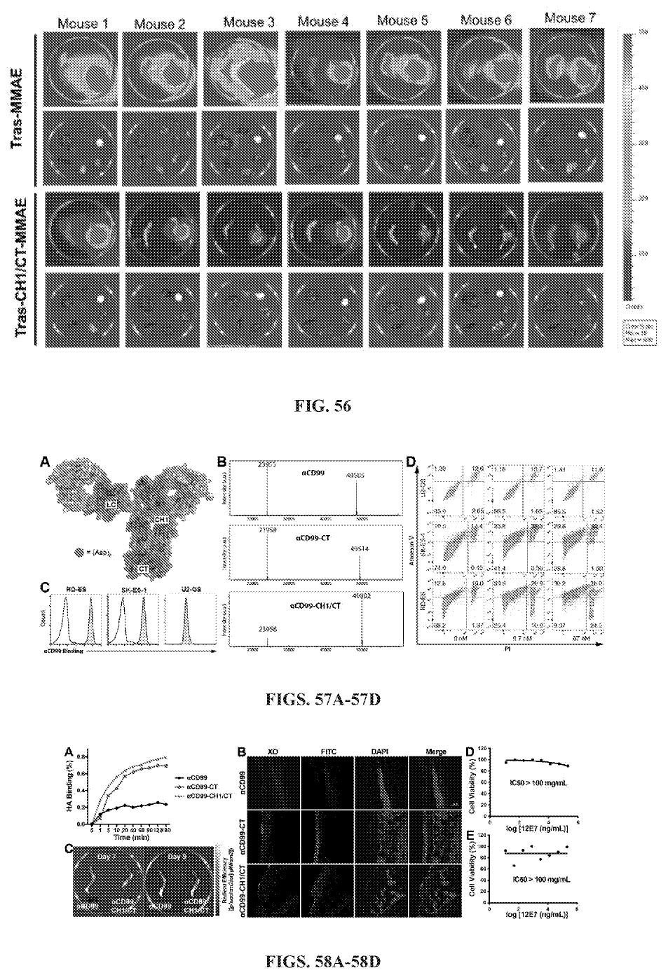

Next, the therapeutic efficacy of bone-targeting ADCs were examined in the xenograft model of bone metastasis. Weekly administration of the Tras-CH1/CT-MMAE led to a significant inhibition of metastatic growth in bone, compared to unmodified Tras-MMAE (FIGS. 37F-H, 51, 52). Furthermore, the bone-targeting ADC showed no apparent toxicity, as indicated by continuous increase of body weight across the different groups during treatment (FIG. 37I). The micro-CT analysis revealed extensive osteolytic bone destructions in both the PBS- and Tras-MMAE-treated group, but not in Tras-CH1/CT-MMAE-treated group (FIGS. 37J, 53). Compared to mice in the PBS- and Tras-MMAE-treated groups, Tras-CH1/CT-MMAE-treated mice exhibited higher bone volume/tissue volume ratio (BV/TV, Figure S46A) and thicker trabecular bone (Tb.Th, FIG. 54B). Histology also confirms the reduction of intratibia tumor burden in Tras-CH1/CT-MMAE mice (FIG. 55). Consistently, bone-targeting ADC also significantly reduced the frequency and size of secondary metastases derived from bone lesions (FIGS. 37K, 56)

Thus, it was demonstrated that adding bone specificity to antibody therapy leads to increased antibody concentrations in the bone metastatic niche, relative to other tissues. This approach yields a targeted therapy against bone metastases as well as against secondary multi-organ metastatic seeding from bone lesions. Using a xenograft model of bone metastasis, it was found that unmodified trastuzumab had poor bone tissue penetration and distribution, thus reducing access of the antibody to its target and limiting its efficacy against cancer cells in the bone microenvironment. Compared with the unmodified antibody, trastuzumab modified with bone-homing peptide sequences (L-Asp₆) exhibited enhanced targeting to sites of bone metastasis. This resulted in improved activity against breast cancer metastasis to the bone and against metastatic seeding from bone lesions to other organs. Most importantly, it was demonstrated that a modified antibody with moderate bone-binding capability had optimal efficacy in vivo. In contrast, an antibody with higher bone-binding capability has suboptimal activity against bone metastases. This may be due to slow release of the latter entity from the bone matrix or to increased electrostatic repulsion resulting from the increased number of peptides with negative charges. The addition of bone specificity to antibody therapy enabled the specific delivery of these agents to the bone. This not only resulted in enhanced therapeutic efficacy, but also reduced adverse side effects associated with systemic distribution of the drug. Thus, a new strategy is provided herein for transitioning antibody-based therapies from antigen-specific to both antigen and tissue-specific, thus providing a promising new avenue for advancing antibody therapy toward clinical translation.

### Example 2—Materials and Methods

Materials. Unless otherwise noted, the chemicals and solvents used were of analytical grade and were used as received from commercial sources. LB agar was ordered from Fisher. Oligonucleotide primers were purchased from Eurofins Genomics (Table 1). Plasmid DNA preparation was carried out with the GenCatch™ Plus Plasmid DNA Miniprep Kit and GenCatch™ Advanced Gel Extraction Kit. PNGase F was purchased from New England Biolabs to remove the glycans before ESI-MS analysis. NuPAGE 4-12% Bis-Tris Gel was purchased from Invitrogen. SDS-PAGE Sample Loading Buffer [6×] was purchased from BIOSCIENCES. PM2500 ExcelBand 3-color Regular Range Protein Marker was purchased from SMOBIO. ExpiCHO Expression Medium, ExpiFectamine CHO Transfection Kit, OptiPRO SFM Complexation Medium were purchased from Thermofisher. Hoechst 33342 (Cat No: H1399) were purchased from Life Technologies™. 3,3-Dioctadecyloxacarbocyanine perchlorate (DiIC18, Cat No: M1197) was purchased from Marker Gene Technologies, Inc.

Cell lines. MDA-MB-361, BT474, SK-BR-3, and MDA-MB-468 cell lines were cultured according to ATCC instructions. Firefly luciferase and RFP labeled MDA-MB-361 cell line was generated as previously described.⁴⁰

SDS-PAGE analysis. The intact and reduced antibody samples were analyzed using invitrogen NuPAGE 4-12% Bis-Tris gel. 20 μL of 0.2 mg/mL antibody samples were mixed with 4 μL sample loading dye before loading into the gel. The gel was running under 160 V in MES buffer for 35 min and stained using commassie blue buffer. The gel images were taken by Amersham Imager 600 and analyzed by ImageQuanTL software.

ESI-MS analysis. The antibodies were analyzed using a single quadrupole mass spectrometer (Agilent: G7129A) coupled with 1260 infinity II Quaternary Pump (Agilent: G7111B). (Column: Pursuit 5 Diphenyl 150×2.0 mm) The elution conditions for antibodies were as follows: mobile phase A=0.1% formic acid water; mobile phase B=0.1% formic acid in acetonitrile; gradient 0-0.1 min, 10-15% B; 0.1-8 min, 15-50% B; 8-8.1 min, 50-10% B; flow rate=0.5 mL/min. The absorbance was measured at 280 nm. Automatic data processing was performed with MassHunter BioConfirm software (Agilent) to analyze the intact and reduced MS spectra.

HA binding assay. Briefly, 1 mg of Tras or bone-targeting antibodies were diluted in 0.5 mL PBS (pH 7.4) in an Eppendorf tube. HA (20 equiv, 20 mg) was suspension in 0.5 mL PBS. Then, the antibodies and HA were mixed with vortex, and the resulting suspension was shaken at 220 rpm at 37° C. Samples without HA were used as controls. After 0.25, 0.5, 1, 2, 3, 6 and 8 hours, the suspension was centrifuged (3000 rpm, 3 min) and the absorbance of the supernatant at 280 nm was measured by Nanodrop. The percent binding to HA was calculated as follow, where OD represents optical density:

$$[ ( O {D}_{without HA} - O {D}_{with HA} ) / ( O {D}_{without HA} ) ] \times 100 \% .$$

In vitro cytotoxicity of Tras, Tras-CT, Tras-CH1/CT and Tras-LC/CH1/CT. BT474, and MDA-MB-468 cells at 2×10³ cells/well into 96-well plates. After 24 h incubation, cells were treated with different concentrations of Tras, Tras-CT, Tras-CH1/CT and Tras-LC/CH1/CT, and then incubated for 4 d. 20 AL of 3-(4,5-dimethylthiazol-2-yl)-2,5-diphenyltetrazolium bromide (MTT) solution (5 mg/mL) was then added to each well and incubated for another 4 h. Medium was aspirated and 150 AL DMSO was added to each well. The absorbance at 565 nm was measured by microplate reader (Infinite M Plex by Tecan) to quantify living cells.

Flow cytometry. Cancer cells (3×10⁵) were incubated with 30 μg/mL Tras and bone-targeting antibodies for 30 min at 4° C. After washing away unbound antibodies, bound antibodies were detected using Fluorescein (FITC) AffiniPure Goat Anti-Human IgG (H+L) (code: 109-095-003, Jackson Immunology) for 30 min at 4° C. Fluorescence intensity was determined using a BD FACSVerse (BD Biosciences).

Determination of K_{d} values. The functional affinity of bone-targeting antibodies for HER2 was determined as reported.⁴¹ Briefly, a total of 2×10⁵ SK-BR-3 or MDA-MB-468 cells were incubated with graded concentrations of Tras, Tras-LC, Tras-CT, Tras-CH1, Tras-LC/CT, Tras-CH1/CT, Tras-LC/CH1 and Tras-LC/CH1/CT for 4 hours on ice. Then, the bound antibody was detected by Fluorescein (FITC) AffiniPure Goat Anti-Human IgG (H+L)

(Jackson Immunology). Cells were analyzed for fluorescence intensity after propidium iodide (Molecular Probes, Eugene, OR) staining. The linear portion of the saturation curve was used to calculate the dissociation constant, KD, using the Lineweaver-Burk method of plotting the inverse of the median fluorescence as a function of the inverse of the antibody concentration. The KD was determined as follows: 1/F=1/Fmax+(K_{d}/Fmax)(1/[Ab]), where F corresponds to the background subtracted median fluorescence and Fmax was calculated from the plot.

Binding to bone cryosections. Nondecalcified long bone sections from C57BL/6 mice were incubated with 50 μg/mL Tras, Tras-CT, Tras-CH1/CT or Tras-LC/CH1/CT, conjugated overnight at 4° C., followed by staining with fluorescein isothiocyanate (FITC)-labeled anti-human IgG for 60 min at room temperature. After washing 3 times with PBS, specimens were incubated for 30 min at 37° C. with Xylenol Orange (XO) (stock: 2 mg/ml, dilute 1:500, dilute buffer: PBS pH 6.5). After three washes with PBS, specimens were stained with Hoechst 33342 (stock 10 mg/ml, dilute 1:2000) for 10 min. Slides were then washed with PBS, air dried, and sealed with PROLONG™ gold anti-fade mountant (from ThermoFisher).

In vivo evaluation of Tras, Tras-CT, Tras-CH1/CT and Tras-LC/CH1/CT. To establish bone metastasis, firefly luciferase and RFP labeled MDA-MB-361 cells (2×10⁵) were inoculated into tibia of 3-4 weeks old female athymic nude mice using para-tibial injection method. 7 days after surgery, mice were ranked/random divided to obtain similar tumor burden in each group. PBS and antibodies (1.0 mg/kg) were injected via retro-orbital injection twice a week. Animals were imaged once a week using IVIS Lumina II (Advanced Molecular Vision), following the recommended procedures and manufacturer's settings. All of the mice were euthanized after blood was collected on day 81, and all the organs (tumor-bearing tibia, heart, liver, spleen, lung, brain and kidney) were collected for further tests.

Ex vivo metastasis-to-metastasis analysis. At the end point, live animals were given D-Luciferin and immediately dissected. The tissues were examined by ex vivo BLI imaging. The whole process organ collection procedure and ex vivo imaging process should be finished within 15 minutes for each mouse.

Bone histology and immunohistochemistry. On day 81, mice were euthanized, and tibiae were harvested, fixed and then decalcified in 12% EDTA for 10 days. The tibiae were embedded in paraffin, and sectioned. Tumor burden was evaluated on hematoxylin and eosin (H&E) sections. Osteoclasts within the tumor and on bone-tumor-interface were counted after staining with Acid Phosphatase, Leukocyte (TRAP) Kit (Sigma). Immunohistochemistry analysis was performed on decalcified paraffin-embedded tissue sections using the HRP/DAB ABC IHC KIT (abcam) following the manufacture's protocol.

Radiographic analysis. Tibiae were dissected, fixed and scanned by microcomputed tomography (micro-CT, Skyscan 1272, Aartselaar, Belgium) at a resolution of 16.16 μm/pixel. Raw images were reconstructed in NReconn and analyzed in CTan (SkyScan, Aartselaar, Belgium) using a region of interest (ROI). Bone parameters analyzed included trabecular thickness (Tb.Th), bone volume fraction (BV/TV), bone mineral density (BMD), and bone surface/bone volume ratio (BS/BV).

Biodistribution. Female athymic nude mice were injected para-tibia with MDA-MB-361 cells (2× 10⁵ cells/animal). After 80 days, Cy7.5 labeled Tras and bone-targeting antibodies were administrated by retro-orbital injection. After 72 h and 120 h injection, the mice were imaged using IVIS. 72 h and 120 h injection, the mice were killed, and major organs including heart, liver, spleen, kidney, lung, and bone tumor tissue were removed. The fluorescence intensity in organs and tumor bearing tibiae were observed using IVIS.

For the wild type mice, Cy7.5 labeled Tras, Tras-CT and Tras-LC/CH1/CT were administered via retro-orbital injection to the C57BL/6 mice. After 48 hours, organs were dissected and imaged using IVIS.

Quantification of TRAP and calcium levels in serum. On day 81, blood was collected by cardiac puncture, and centrifuged for 15 min at 3,000 rpm to obtain the serum. The concentration of osteoclast-derived TRACP 5b was measured by using a Mouse ACP5/TRAP ELISA Kit (catalog number IT5180, GBiosciences). Serum calcium levels were determined colorimetrically using a calcium detection kit (catalog number DICA-500, Bioassays).

Statistical methods. Data are presented as means plus or minus SEM and statistically analyzed using GraphPad Prism software version 6 (GraphPad software, San Diego, CA). Two-way ANOVA followed by Sidak's multiple comparisons was used for all data collected over a time course. One-way ANOVA followed by Tukey's multiple comparisons was used for Micro-CT data. Unpaired Student's 1-test was used for multi-organ metastasis data. P<0.05 was considered to represent statistical significance.

Plasmid construction. pCDNA-Tras-LC: The 6D gene was inserted into the LC chain of Tras sequence (bordering residues A153, Q155) by PCR using primers RG03, RG04, RG07 and RG08 and pCDNA-Tras as template. The gene fragments were annealed by using Gibson assembly method.

pCDNA-Tras-CH1: The 6D gene was inserted into the CH1 chain of Tras sequence (bordering residues A165, G169) by PCR using primers RG05, RG06, RG07 and RG008 and pCDNA-Tras as template. The gene fragments were annealed by using Gibson assembly method.

pCDNA-Tras-CT: The 6D gene was inserted into the C-terminus of Tras sequence (bordering residue G449) by PCR using primers RG01, RG02, RG07 and RG008 and pCDNA-Tras as template. The gene fragments were annealed by using Gibson assembly method.

pCDNA-Tras-LCCH1/LCCT/CH1CT/LCCH1CT-6D: The 6D genes were inserted into the Tras sequence by using the primers mentioned above and the constructed plasmids as templates. The gene fragments were annealed by using Gibson assembly method.

TABLE 1
DNA Oligomers.

| oligonucleotide | Sequence (5′-3′) |
|---|---|
| RG01 (SEQ ID | GATGATGACGACGACGACTGAGCGGCCGCTCG |
| NO: 17) | |
| RG02 (SEQ ID | CTCAGTCGTCGTCGTCATCATCACCCGGAGACAGGGAGAG |
| NO: 18) | |
| RG03 (SEQ ID | GATGATGATGACGACGATCAATCGGGTAACTCCCAGG |
| NO: 19) | |
| RG04 (SEQ ID | CCGATTGATCGTCGTCATCATCATCGGCGTTATCCACCTTCCAC |
| NO: 20) | |
| RG05 (SEQ ID | GACGACGATGACGACGATGGCGTGCACACCTTCCC |
| NO: 21) | |
| RG06 (SEQ ID | GCACGCCATCGTCGTCATCGTCGTCgGCGCCTGAGTTCCACGA |
| NO: 22) | C |
| RG07 (SEQ ID | GTCGAACTTTTCGATCAGAAACTTCTCGA |
| NO: 23) | |
| RG08 (SEQ ID | TCGAGAAGTTTCTGATCGAAAAGTTCGAC |
| NO: 24) | |

Expression and purification of antibody mutants. Tras and 6D-inserted mutants were expressed by ExpiCHO-S cells following Thermofisher's ExpiCHO expression protocol. Cells were grown and subcultured in a 37° C. incubator with >80% relative humidity and 8% CO₂ on an orbital shaker platform (125 rpm) until cultures reach a density of 4×10⁶-6×10⁶ viable cells/mL. Before transfection, ExpiFectamine CHO/plasmid DNA complexes were prepared and incubated at room temperature for 5 min, and then were slowly added to the cell culture. After 18-22 hours post-transfection, ExpiFectamine CHO Enhancer and ExpiCHO Feed were added to the cell culture. After 12 days expression, the secreted antibodies were harvested by centrifugation at 9000 rpm for 30 min and purified on Protein G resin following manufacturer's instructions. Before injection, each antibody sample was buffer-exchanged into PBS via a PD-10 desalt column and the concentration was measured using a NanoDrop Lite (Thermofisher). All the antibody samples were characterized by ESI-MS and SDS-PAGE analysis.

TABLE 2
Absorption of 6D-inserted mutants bind to HA.

| Antibodies | Percentage of the bound fraction |
|---|---|
| Tras | 23.9 ± 4.7 |
| Tras-LC | 68.3 ± 4.6 |
| Tras-CH1 | 63.9 ± 1.2 |
| Tras-CT | 71.9 ± 2.5 |
| Tras-LC/CT | 59.7 ± 4.4 |
| Tras-CH1/CT | 81.1 ± 3.1 |
| Tras-LC/CH1/CT | 80.3 ± 1.1 |

The antibodies (1 mg/ml) were incubated with 20 mg/ml hydroxyapatite at 37° C. for 9 h. Percentages of the hydroxyapatite-bound fraction are shown (mean ± SEM).

TABLE 3
Potency and cell-surface reactivity of Tras, Tras-CT Tras-LC, Tras-CH1, Tras-LC/CH1, Tras-
LC/CT, Tras-CH1/CT and Tras-LC/CH1/CT against breast cancer epithelial cell lines.

| | | MFI increase (fold) | | | | | | | |
|---|---|---|---|---|---|---|---|---|---|
| | Her2 | | Tras- | Tras- | Tras- | Tras- | Tras- | Tras- | Tras- |
| Cell Line | Expression | Tras | CT | LC | CH1 | LC/CH1 | LC/CT | CH1/CT | LC/CH1/CT |
| SK-BR-3 | 3+ | 65.11 | 78.72 | 83.54 | 83.94 | 7.53 | 57.75 | 78.60 | 65.55 |
| MDA-MB-468 | 0 | 1.41 | 1.87 | 1.90 | 1.56 | 1.45 | 1.16 | 1.92 | 1.24 |

Abbreviations: MFI, median fluorescence intensity. Binding was determined as the mean fold increase in median fluorescence over the PBS control.

TABLE 4
Comparison of different treatment groups in multiple assays.

| | | | Osteoclast activity | | | | |
|---|---|---|---|---|---|---|---|
| | BLI | | Osteoclast | Serum | Serum | Body weight | |
| Treatment | Progression^{a} | Fold-increase^{b} | number^{c} | TRACP 5b^{d} | Calcium^{e} | Progression^{f} | Survival^{g} |
| Control vs Tras | ** | ns | **** | ns | ns | ns | ns |
| Control vs Tras-CT | **** | **** | **** | * | ns | ns | ** |
| Control vs Tras-CH1/CT | **** | **** | **** | ** | * | ns | *** |
| Control vs Tras-LC/CH1/CT | **** | **** | **** | * | ns | ns | * |
| Tras vs Tras-CT | ns | **** | ns | ns | ns | ns | ns |
| Tras vs Tras-CH1/CT | ** | **** | *** | ns | ns | ns | * |
| Tras vs Tras-LC/CH1/CT | ns | **** | ns | ns | ns | ns | ns |

Abbreviations: BLI, bioluminescence imaging; TRAP, tartrate-resistant acid phosphatase.
^{a}Signal intensity of BLI in whole body over the course of the experiment.
^{b}Signal intensity fold-increase of BLI after treatment (BLI of day 80/BLI of day 1).
^{c}Osteoclast number measurement from TRAP-stained tibia sections at the end of experiment.
^{d}Serum TRACP 5b concentration at the end of experiment.
^{e}Serum calcium concentration at the end of experiment.
^{f}Body weight progression over the course of the experiment.
^{g}The mice BLI intensity over 10⁷ was considered to reach the endpoint.
^{a, b}were analyzed statistically by using a two-way repeated-measure ANOVA followed by Sidak's multiple comparisons test.
^{c, d, e, f}were analyzed by using a one-way ANOVA followed by Tukey's multiple comparisons test.
^{g}was analyzed by using a log-rank test.
**** P < 0.0001,
*** P < 0.001,
** P < 0.01,
* P < 0.05,
ns represents P > 0.05.

TABLE 5
Comparison microCT parameters from different treatment groups.

| | Trabecular bone parameters | | | | |
|---|---|---|---|---|---|
| Groups | BV | BV/TV | BS/BV | BMD | Tb. Th |
| Control vs Tras | ns | ns | ns | ns | ns |
| Control vs Tras-CT | ** | ns | ns | * | ** |
| Control vs Tras- | **** | ** | ** | ** | ** |
| CH1/CT | | | | | |
| Control vs Tras- | ** | * | ns | ns | ** |
| LC/CH1/CT | | | | | |
| Tras vs Tras-CT | ns | ns | ns | ** | ns |
| Tras vs Tras- | *** | * | * | *** | * |
| CH1/CT | | | | | |
| Tras vs Tras- | * | ns | ns | * | ns |
| LC/CH1/CT | | | | | |

Abbreviations:
BV: bone volume;
BV/TV: bone volume/tissue volume ratio;
BS/BV: bone surface/bone volume ratio;
BMD: bone mineral density;
Tb. Th: trabecular thickness.
Data was presented as mean ± sem.
The microCT data were analyzed by using a one-way ANOVA followed by Tukey's multiple comparisons test.
****P < 0.0001,
***P < 0.001,
**P < 0.001,
*P < 0.05,
ns represents P > 0.05.

### Example 3-Engineering αCD99 Antibody for Ewing Sarcoma with Bone-Homing Peptides

Peptides were used to engineer αCD99 antibodies for bone targeting. ES tumors have much higher CD99 expression levels than normal cells. To achieve specific targeting of high CD99-expressing ES tumors but not normal human cells, a full-length antibody was first expressed with relatively low CD99 affinity (clone: 12E7, K_{D}=10 nM, FIG. 57). The antibody clone has been recently used in the clinical study and demonstrated an excellent safety profile. It was demonstrated that this purified αCD99 antibody can specifically bind to CD99-positive RD-ES, SK-ES-1 cells, but not CD99-negative U2-OS osteosarcoma cells (FIG. 57C). Furthermore, 12E7 antibody treatment induced apoptosis in more than 60% and 70% of RD-ES and SK-ES-1 cells, respectively (FIG. 57D). The in vivo therapeutic efficacy of this αCD99 antibody was also validated by us in xenograft models (FIG. 59).

To prepare antibodies targeting bone resorption niche, bone-homing peptide, L-Asp₆ peptide, was introduced into permissive internal sites in αCD99 antibody light chain (LC, A153), heavy chain (CH1, A165), and C-terminus (CT, G449) to yield bone-targeting αCD99 antibodies (FIGS. 57A and B). These internal sites were shown to bear additional peptide insertion by screening an antibody peptide-placement library. αCD99 was prepared with one L-Asp₆ peptide (αCD99-CT, FIG. 57B).

Antibodies were prepared with different numbers of L-Asp₆ peptides and their bone targeting activity was evaluated, L-Asp₆ peptides were cloned into two (CH1 and CT) and three permissive internal sites (LC, CH1, and CT) to yield the αCD99 antibody mutants with two and three L-Asp₆ peptides, respectively. The successful preparation of αCD99-CH1/CT has been confirmed by ESI-MS (FIG. 57B).

The bone affinity difference was evaluated among the bone-targeting antibodies. αCD99, αCD99-CT, αCD99-CH1/CT, and αCD99-LC/CH1/CT, antibodies were incubated with hydroxyapatite/native bone. The introduction of L-Asp₆ sequence into antibodies can significantly enhance their bone-targeting ability. As shown in FIG. 58A, the wild type αCD99 exhibited a slight binding affinity with HA, while 80%-95% of antibodies containing L-Asp₆ peptide sequences bound to HA within 4 h. In general, bone-targeting antibodies with more bone-homing peptides exhibit better HA binding affinity (FIG. 58A). Furthermore, FITC-labeled αCD99, αCD99-CT, and αCD99-CH1/CT mutants were further used to stain nondecalcified bone sections from C57BL/6 mice as shown in FIG. 58B. The FITC signal was observed for the section stained with the αCD99-CT, αCD99-CH1/CT antibody mutants, but not wild type αCD99 (FIG. 58B).

No cytotoxicity of 12E7 antibody was observed with human macrophages and endothelial cells (FIGS. 58D and E). To examine the feasibility of using bone-specific antibodies to treat ES, the impact of αCD99 antibody engineered with L-Asp₆ sequences on orthotopic models was examined using established cell lines and patient-derived xenograft models (FIGS. 59A-C). To demonstrate that L-Asp₆ sequence modification can enhance the bone binding affinity for antibodies, a xenograft nude mice model was established. 5×10⁵ SK-ES-1 (CD99+++) ES cells labeled were inoculated with firefly luciferase and red fluorescent protein into the right leg of nude mice via intra-tibial injection (FIG. 58C). NIR dye-modified wild type αCD99 (1 mg/kg retro-orbitally in sterile PBS) and αCD99-CH1/CT, (same regimen as Tras) were admitted using retro-orbital injection. Seven and 9 days after administration, the major organs were removed and analyzed using a Caliper IVIS Lumina II in vivo imager. The fluorescence intensity in bone tissue was higher for the antibodies with L-Asp₆ peptide sequence than for wild type αCD99 (FIG. 58C).

To demonstrate the ability of bone-targeting αCD99 to inhibit in vivo ES development in the bone and soft tissues a xenograft model was established to study the in vivo therapeutic efficacy of αCD99 against ES tumors in the bone. 5×10⁵ SK-ES-1 cells labeled with firefly luciferase and red fluorescent protein were inoculated into the nude mice via intra-tibial injection. As shown in FIGS. 59A and 59B, whole-body bioluminescence imaging (BLI) signals suggested that treatments with bone-targeting &CD99 resulted in more significant inhibition of ES tumor progression, compared to that seen in wild type αCD99-treated mice (FIGS. 59A-C).

To evaluate the efficacy of bone-targeting αCD99 for the treatment of nonbone ES tumors, intramuscular injection was used to inject 5×10⁵ SK-ES-1 cells into the close proximity to the tibia. Bone-targeting αCD99 antibody exhibited a superb and similar efficacy for the treatment of ES located in soft tissues compared with wild type αCD99 (FIGS. 59A-C).

### Example 4—Bone-Targeting IL-6

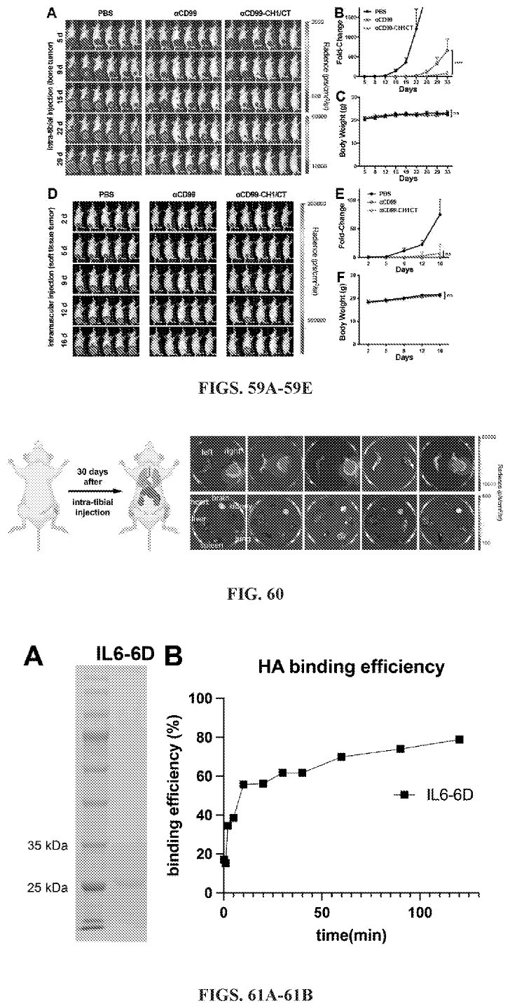

Further, bone-targeting interleukin-6 (IL6) was generated by inserting six aspartic acids at the C-terminus of IL-6 (FIG. 61A). The resulting bone-targeting IL6 (IL6-6D) exhibited enhanced binding affinity to the hydroxyapatite structure (FIG. 61B).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
(1) Tran, L.; Xiao, J.-F.; Agarwal, N.; Duex, J. E.; Theodorescu, D. Advances in Bladder Cancer Biology and Therapy. Nat Rev Cancer 2020.
(2) Scott, A. M.; Wolchok, J. D.; Old, L. J. Antibody Therapy of Cancer. Nat Rev Cancer 2012, 12 (4), 278-287.
(3) Marston, H. D.; Paules, C. I.; Fauci, A. S. Monoclonal Antibodies for Emerging Infectious Diseases—Borrowing from History. N Engl J Med 2018, 378 (16), 1469-1472.
(4) Lu, L. L.; Suscovich, T. J.; Fortune, S. M.; Alter, G. Beyond Binding: Antibody Effector Functions in Infectious Diseases. Nat Rev Immunol 2018, 18 (1), 46-61.
(5) Loupy, A.; Lefaucheur, C. Antibody-Mediated Rejection of Solid-Organ Allografts. N Engl J Med 2018, 379 (12), 1150-1160.
(6) Dai, Z.; Zhang, X.-N.; Nasertorabi, F.; Cheng, Q.; Li, J.; Katz, B. B.; Smbatyan, G.; Pei, H.; Louie, S. G.; Lenz, H.-J.; Stevens, R. C.; Zhang, Y. Synthesis of Site-Specific Antibody-Drug Conjugates by ADP-Ribosyl Cyclases. Sci. Adv. 2020, 6 (23), eaba6752.
(7) Chaganty, B. K. R.; Qiu, S.; Gest, A.; Lu, Y.; Ivan, C.; Calin, G. A.; Weiner, L. M.; Fan, Z. Trastuzumab Upregulates PD-L1 as a Potential Mechanism of Trastuzumab Resistance through Engagement of Immune Effector Cells and Stimulation of IFNγ Secretion. Cancer Letters 2018, 430, 47-56.
(8) Maruani, A.; Szijj, P. A.; Bahou, C.; Nogueira, J. C. F.; Caddick, S.; Baker, J. R.; Chudasama, V. A Plug-and-Play Approach for the De Novo Generation of Dually Functionalized Bispecifics. Bioconjugate Chem. 2020, 31 (3), 520-529.
(9) Zhang, Y.; Wang, D.; Welzel, G.; Wang, Y.; Schultz, P. G.; Wang, F. An Antibody CDR3-Erythropoietin Fusion Protein. ACS Chem. Biol. 2013, 8 (10), 2117-2121.
(10) Cruz, E.; Kayser, V. Monoclonal Antibody Therapy of Solid Tumors: Clinical Limitations and Novel Strategies to Enhance Treatment Efficacy. Biologics 2019, 13, 33-51.
(11) Baker, J. H. E.; Kyle, A. H.; Reinsberg, S. A.; Moosvi, F.; Patrick, H. M.; Cran, J.; Saatchi, K.; Häfeli, U.; Minchinton, A. I. Heterogeneous Distribution of Trastuzumab in HER2-Positive Xenografts and Metastases: Role of the Tumor Microenvironment. Clin Exp Metastasis 2018, 35 (7), 691-705.
(12) Pallasch, C. P.; Leskov, I.; Braun, C. J.; Vorholt, D.; Drake, A.; Soto-Feliciano, Y. M.; Bent, E. H.; Schwamb, J.; Iliopoulou, B.; Kutsch, N.; van Rooijen, N.; Frenzel, L. P.; Wendtner, C. M.; Heukamp, L.; Kreuzer, K. A.; Hallek, M.; Chen, J.; Hemann, M. T. Sensitizing Protective Tumor Microenvironments to Antibody-Mediated Therapy. Cell 2014, 156 (3), 590-602.
(13) Kennecke, H.; Yerushalmi, R.; Woods, R.; Cheang, M. C. U.; Voduc, D.; Speers, C. H.; Nielsen, T. O.; Gelmon, K. Metastatic Behavior of Breast Cancer Subtypes. J. Clin. Oncol. 2010, 28 (20), 3271-3277.
(14) Spadazzi et al.—2021—Trefoil Factor-1 Upregulation in Estrogen-Receptor.Pdf.
(15) Riggio, A. I.; Varley, K. E.; Welm, A. L. The Lingering Mysteries of Metastatic Recurrence in Breast Cancer. Br J Cancer 2021, 124 (1), 13-26.
(16) Gullo, G.; Zuradelli, M.; Sclafani, F.; Santoro, A.; Crown, J. Durable Complete Response Following Chemotherapy and Trastuzumab for Metastatic HER2-Positive Breast Cancer. Ann. Oncol. 2012, 23 (8), 2204-2205.
(17) Fujisawa, R.; Wada, Y.; Nodasaka, Y.; Kuboki, Y. Acidic Amino Acid-Rich Sequences as Binding Sites of Osteonectin to Hydroxyapatite Crystals. Biochimica et Biophysica Acta (BBA)—Protein Structure and Molecular Enzymology 1996, 1292 (1), 53-60.
(18) Liu, J.; Dang, L.; Li, D.; Liang, C.; He, X.; Wu, H.; Qian, A.; Yang, Z.; Au, D. W. T.; Chiang, M. W. L.; Zhang, B.-T.; Han, Q.; Yue, K. K. M.; Zhang, H.; Lv, C.; Pan, X.; Xu, J.; Bian, Z.; Shang, P.; Tan, W.; Liang, Z.; Guo, B.; Lu, A.; Zhang, G. A Delivery System Specifically Approaching Bone Resorption Surfaces to Facilitate Therapeutic Modulation of MicroRNAs in Osteoclasts. Biomaterials 2015, 52, 148-160.
(19) Miller, S. C.; Pan, H.; Wang, D.; Bowman, B. M.; Kopecková, P.; Kopecek, J. Feasibility of Using a Bone-Targeted, Macromolecular Delivery System Coupled with Prostaglandin E (1) to Promote Bone Formation in Aged, Estrogen-Deficient Rats. Pharm. Res. 2008, 25 (12), 2889-2895.
(20) Zhang, Y.; Wei, L.; Miron, R. J.; Shi, B.; Bian, Z. Anabolic Bone Formation via a Site-Specific Bone-Targeting Delivery System by Interfering with Semaphorin 4D Expression. J. Bone Miner. Res. 2015, 30 (2), 286-296.
(21) Sekido, T.; Sakura, N.; Higashi, Y.; Miya, K.; Nitta, Y.; Nomura, M.; Sawanishi, H.; Morito, K.; Masamune, Y.; Kasugai, S.; Yokogawa, K.; Miyamoto, K.-I. Novel Drug Delivery System to Bone Using Acidic Oligopeptide: Pharmacokinetic Characteristics and Pharmacological Potential. Journal of Drug Targeting 2001, 9 (2), 111-121.
(22) Wang, D.; Miller, S. C.; Shlyakhtenko, L. S.; Portillo, A. M.; Liu, X.-M.; Papangkorn, K.; Kopecková, P.; Lyubchenko, Y.; Higuchi, W. I.; Kopecek, J. Osteotropic Peptide That Differentiates Functional Domains of the Skeleton. Bioconjug. Chem. 2007, 18 (5), 1375-1378.
(23) Dang, L.; Liu, J.; Li, F.; Wang, L.; Li, D.; Guo, B.; He, X.; Jiang, F.; Liang, C.; Liu, B.; Badshah, S. A.; He, B.; Lu, J.; Lu, C.; Lu, A.; Zhang, G. Targeted Delivery Systems for Molecular Therapy in Skeletal Disorders. Int J Mol Sci 2016, 17 (3).
(24) Kingsley, L. A.; Fournier, P. G. J.; Chirgwin, J. M.; Guise, T. A. Molecular Biology of Bone Metastasis. Mol. Cancer Ther. 2007, 6 (10), 2609-2617.
(25) Ren, G.; Esposito, M.; Kang, Y. Bone Metastasis and the Metastatic Niche. J Mol Med 2015, 93 (11), 1203-1212.
(26) Ell, B.; Kang, Y. SnapShot: Bone Metastasis. Cell 2012, 151 (3), 690-690.e1.
(27) Drake, P. M.; Albers, A. E.; Baker, J.; Banas, S.; Barfield, R. M.; Bhat, A. S.; de Hart, G. W.; Garofalo, A. W.; Holder, P.; Jones, L. C.; Kudirka, R.; McFarland, J.; Zmolek, W.; Rabuka, D. Aldehyde Tag Coupled with HIPS Chemistry Enables the Production of ADCs Conjugated Site-Specifically to Different Antibody Regions with Distinct in vivo Efficacy and PK Outcomes. Bioconjug. Chem. 2014, 25 (7), 1331-1341.
(28) Weigelt, B.; Peterse, J. L.; van′t Veer, L. J. Breast Cancer Metastasis: Markers and Models. Nat Rev Cancer 2005, 5 (8), 591-602.
(29) Weilbaecher, K. N.; Guise, T. A.; McCauley, L. K. Cancer to Bone: A Fatal Attraction. Nat Rev Cancer 2011, 11 (6), 411-425.
(30) Coleman, R.; Smith, P.; Rubens, R. Clinical Course and Prognostic Factors Following Bone Recurrence from Breast Cancer. Br J Cancer 1998, 77 (2), 336-340.
(31) Coleman, R.; Rubens, R. The Clinical Course of Bone Metastases from Breast Cancer. Br J Cancer 1987, 55 (1), 61-66.
(32) Wang, H.; Tian, L.; Liu, J.; Goldstein, A.; Bado, I.; Zhang, W.; Arenkiel, B. R.; Li, Z.; Yang, M.; Du, S.; Zhao, H.; Rowley, D. R.; Wong, S. T. C.; Gugala, Z.; Zhang, X. H.-F. The Osteogenic Niche Is a Calcium Reservoir of Bone Micrometastases and Confers Unexpected Therapeutic Vulnerability. Cancer Cell 2018, 34 (5), 823-839.e7.
(33) Wang, H.; Yu, C.; Gao, X.; Welte, T.; Muscarella, A. M.; Tian, L.; Zhao, H.; Zhao, Z.; Du, S.; Tao, J.; Lee, B.; Westbrook, T. F.; Wong, S. T. C.; Jin, X.; Rosen, J. M.; Osborne, C. K.; Zhang, X. H.-F. The Osteogenic Niche Promotes Early-Stage Bone Colonization of Disseminated Breast Cancer Cells. Cancer Cell 2015, 27 (2), 193-210.
(34) Yu, C.; Wang, H.; Muscarella, A.; Goldstein, A.; Zeng, H.-C.; Bae, Y.; Lee, B. H. I.; Zhang, X. H.-F. Intra-Iliac Artery Injection for Efficient and Selective Modeling of Microscopic Bone Metastasis. J Vis Exp 2016, No. 115.
(35) Hansel-2010—The Safety and Side Effects of Monoclonal Antibodi.Pdf.
(36) Marty, M.; Cognetti, F.; Maraninchi, D.; Snyder, R.; Mauriac, L.; Tubiana-Hulin, M.; Chan, S.; Grimes, D.; Antón, A.; Lluch, A.; Kennedy, J.; O'Byrne, K.; Conte, P.; Green, M.; Ward, C.; Mayne, K.; Extra, J.-M. Randomized Phase II Trial of the Efficacy and Safety of Trastuzumab Combined with Docetaxel in Patients with Human Epidermal Growth Factor Receptor 2-Positive Metastatic Breast Cancer Administered as First-Line Treatment: The M77001 Study Group. J. Clin. Oncol. 2005, 23 (19), 4265-4274.
(37) Valero, V.; Forbes, J.; Pegram, M. D.; Pienkowski, T.; Eiermann, W.; von Minckwitz, G.; Roche, H.; Martin, M.; Crown, J.; Mackey, J. R.; Fumoleau, P.; Rolski, J.; Mrsic-Krmpotic, Z.; Jagiello-Gruszfeld, A.; Riva, A.; Buyse, M.; Taupin, H.; Sauter, G.; Press, M. F.; Slamon, D. J. Multicenter Phase III Randomized Trial Comparing Docetaxel and Trastuzumab with Docetaxel, Carboplatin, and Trastuzumab as First-Line Chemotherapy for Patients with HER2-Gene-Amplified Metastatic Breast Cancer (BCIRG 007 Study): Two Highly Active Therapeutic Regimens. J. Clin. Oncol. 2011, 29 (2), 149-156.
(38) Nahta, R.; Yu, D.; Hung, M.-C.; Hortobagyi, G. N.; Esteva, F. J. Mechanisms of Disease: Understanding Resistance to HER2-Targeted Therapy in Human Breast Cancer. Nat Clin Pract Oncol 2006, 3 (5), 269-280.
(39) Schmid, P.; Adams, S.; Rugo, H. S.; Schneeweiss, A.; Barrios, C. H.; Iwata, H.; Diéras, V.; Hegg, R.; Im, S.-A.; Shaw Wright, G.; Henschel, V.; Molinero, L.; Chui, S. Y.; Funke, R.; Husain, A.; Winer, E. P.; Loi, S.; Emens, L. A. Atezolizumab and Nab-Paclitaxel in Advanced Triple-Negative Breast Cancer. New England Journal of Medicine 2018, 379 (22), 2108-2121.
(40) Wang, H.; Tian, L.; Goldstein, A.; Liu, J.; Lo, H.-C.; Sheng, K.; Welte, T.; Wong, S. T. C.; Gugala, Z.; Stossi, F.; Zong, C.; Li, Z.; Mancini, M. A.; Zhang, X. H.-F. Bone-in-Culture Array as a Platform to Model Early-Stage Bone Metastases and Discover Anti-Metastasis Therapies. Nat Commun 2017, 8 (1), 15045.
(41) Dillon, R. L.; Chooniedass, S.; Premsukh, A.; Adams, G. P.; Entwistle, J.; MacDonald, G. C.; Cizeau, J. Trastuzumab-DeBouganin Conjugate Overcomes Multiple Mechanisms of T-DM1 Drug Resistance: Journal of Immunotherapy 2016, 39 (3), 117-126.

What is claimed:

1. A bone-targeting antibody engineered to comprise at least one bone-homing peptide which selectively binds to bone hydroxyapatite (HA).

2. The antibody of claim 1, wherein the at least one bone-homing peptide is inserted at a permissive internal site of the antibody.

3. The antibody of claim 1 or 2, wherein the at least one bone-homing peptide is inserted at the light chain (LC), heavy chain (CH1), and/or C-terminus (CT) of the antibody.

4. The antibody of any of claims 1-3, wherein the antibody comprises two, three, or four bone-homing peptides.

5. The antibody of any of claims 1-4, wherein the bone-homing peptide is L-Asp₃, L-Asp₄, L-Asp₅, L-Asp₆, L-Asp₇, L-Asp₈, L-Asp₉, or L-Asp₁₀.

6. The antibody of any of claims 1-4, wherein the bone-homing peptide is L-Asp₆.

7. The antibody of any of claims 1-6, wherein the antibody is a monoclonal antibody, bispecific antibody, Fab′, a F(ab′)₂, a F(ab′)₃, a monovalent scFv, a bivalent scFv, a single domain antibody, or nanobody.

8. The antibody of any of claims 1-7, wherein the antibody is an immune checkpoint inhibitor.

9. The antibody of any of claims 1-8, wherein the antibody is an anti-HER2 antibody, anti-CD99 antibody, anti-IGF-IR antibody, anti-PD-L1, anti-PD-1, anti-CTLA4-antibody, anti-Siglec-2 antibody, anti-Siglec-3 antibody, anti-Siglec-5 antibody, anti-Siglec-6 antibody, anti-Siglec-7 antibody, anti-Siglec-8 antibody, anti-Siglec9 antibody, anti-Siglec-10 antibody, anti-Siglec-11 antibody, anti-Siglec-15 antibody, anti-RANKL antibody, anti-EGFR antibody, anti-VEGFR antibody, anti-CD20 antibody, anti-CD22 antibody, anti-CD52 antibody, anti-Trop-2 antibody, anti-CD30 antibody, anti-CD152 antibody, anti-IL-6R antibody, anti-GD2 antibody, or anti-TGFβ antibody.

10. The antibody of any of claims 1-8, wherein the antibody is an anti-CD99 antibody.

11. The antibody of any of claims 1-9, wherein the antibody is conjugated to a drug.

12. The antibody of claim 11, wherein the drug is monomethyl auristatin E (MMAE), ozogamicin, tiuxetan, vedotin, pasudotoxtdfx, vedotin, mafodotin, calicheamicin, maytansine, or deruxtecan.

13. The antibody of claim 11, wherein the drug is an anti-mitotic drug.

14. The antibody of claim 13, wherein the anti-mitotic drug is monomethyl auristatin E (MMAE).

15. The antibody of any of claims 1-9, wherein the antibody is an anti-HER2 antibody.

16. The antibody of any of claims 1-15, wherein the antibody is trastuzumab (Herceptin), pertuzumab (Perjeta), or atezolizumab.

17. The antibody of any of claims 1-16, wherein the antibody is trastuzumab.

18. The antibody of claim 17, wherein the trastuzumab is conjugated to MMAE.

19. The antibody of any of claims 1-17, wherein the bone-homing peptide is inserted at residue A153, A165, and/or G449 of trastuzumab.

20. The antibody of any of claims 1-19, wherein the antibody has an amino acid sequence having at least 90% sequence identity to Tras-LC (SEQ ID NOs: 3-4), Tras-CH1 (SEQ ID NOs: 5-6), Tras-CT (SEQ ID NOs: 7-8), Tras-LC/CH1 (SEQ ID NOs: 9-10), Tras-LC/CT (SEQ ID NOs: 11-12), Tras-CH1/CT (SEQ ID NOs: 13-14), or Tras-LC/CH1/CT (SEQ ID NOs: 15-16).

21. The antibody of any of claims 1-20, wherein the antibody has an amino acid sequence having at least 95% sequence identity to Tras-LC (SEQ ID NOs: 3-4), Tras-CH1 (SEQ ID NOs: 5-6), Tras-CT (SEQ ID NOs: 7-8), Tras-LC/CH1 (SEQ ID NOs: 9-10), Tras-LC/CT (SEQ ID NOs: 11-12), Tras-CH1/CT (SEQ ID NOs: 13-14), or Tras-LC/CH1/CT (SEQ ID NOs: 15-16).

22. The antibody of any of claims 1-21, wherein the antibody comprises Tras-LC (SEQ ID NOs: 3-4), Tras-CH1 (SEQ ID NOs: 5-6), Tras-CT (SEQ ID NOs: 7-8), Tras-LC/CH1 (SEQ ID NOs: 9-10), Tras-LC/CT (SEQ ID NOs: 11-12), Tras-CH1/CT (SEQ ID NOs: 13-14), or Tras-LC/CH1/CT (SEQ ID NOs: 15-16).

23. The antibody of any of claims 1-22, wherein the bone-targeting antibody has increased binding affinity to HA as compared to an antibody that does not comprise the bone-homing peptide.

24. The antibody of any of claims 1-23, wherein the bone-targeting antibody has two-fold to three-fold higher binding affinity to HA as compared to an antibody that does not comprise the bone-homing peptide.

25. A method of treating or preventing bone disease in a subject comprising administering to the subject an effective amount of a bone-targeting antibody of any of claims 1-23.

26. The method of claim 25, wherein the subject has bone cancer or bone metastasis.

27. The method of claim 26, wherein the bone cancer is Ewing sarcoma, osteosarcoma, or chondrosarcoma.

28. The method of claim 26, wherein the bone metastasis is from breast cancer, myeloma, renal cancer, lung cancer, prostate cancer, thyroid cancer, or bladder cancer.

29. The method of any of claims 25-28, wherein the breast cancer is triple-negative breast cancer.

30. The method of any of claims 28-29, wherein the breast cancer is HER2-negative breast cancer.

31. The method of any of claims 28-30, wherein the breast cancer is HER2-positive breast cancer.

31.1 The method of any of claims 25-31, wherein the bone disease is wherein the bone disease is osteoporosis, osteomalacia, periodontitis, rheumatoid arthritis, metabolic bone disease, a parathyroid disorder, steroid-induced osteoporosis, chemotherapy-induced bone loss, pre-menopausal bone loss, fragility and recurrent fractures, renal osteodystrophy, bone infections, or Paget's disease.

32. The method of any of claims 25-34, wherein the bone-targeting antibody results in increased concentration of therapeutic antibody at the bone tumor niche, inhibits cancer development in the bone, and/or limits secondary metastases to other organs.

33. The method of any of claims 25-32, wherein the bone-targeting antibody results in decreased micrometastasis-induced osteolyic lesions.

34. The method of any of claims 25-33, wherein the method comprises further administering an additional anti-cancer therapy.

35. The method of claim 34, wherein the additional anti-cancer therapy comprises surgery, chemotherapy, radiation therapy, hormonal therapy, immunotherapy or cytokine therapy.

36. The method of claim 34, wherein the additional anti-cancer therapy comprises immunotherapy or chemotherapy.

37. Use of a bone-targeting antibody of any of claims 1-23 for the treatment or prevention of bone tumors in a subject with cancer.

38. The use of claim 37, wherein the subject has bone cancer or bone metastasis.

39. The use of claim 38, wherein the bone cancer is Ewing sarcoma, osteosarcoma, or chondrosarcoma.

40. The use of claim 38, wherein the bone metastasis is from breast cancer, myeloma, renal cancer, lung cancer, prostate cancer, thyroid cancer, or bladder cancer.

41. A method for engineering a bone-targeting antibody of any of claims 1-23 comprising inserting at least one bone-homing peptide at a permissive internal site of said antibody.

42. The method of claim 41, wherein the antibody comprises two, three, or four bone-homing peptides.

43. The method of claim 41 or 43, wherein the bone-homing peptide is L-Asp₆.

44. A bone-targeting composition comprising one or more polypeptides engineered to comprise at least one bone-homing peptide which selectively binds to bone hydroxyapatite (HA).

45. The antibody of claim 44, wherein the at least one bone-homing peptide is inserted at a permissive internal site of the polypeptide.

46. The antibody of claim 44 or 45, wherein the at least one bone-homing peptide is inserted at the C-terminus or N-terminus of the polypeptide.

47. The antibody of any of claims 44-46, wherein the polypeptide comprises two, three, or four bone-homing peptides.

48. The antibody of any of claims 44-47, wherein the bone-homing peptide is L-Asp₃, L-Asp₄, L-Asp₅, L-Asp₆, L-Asp₇, L-Asp₈, L-Asp₉, or L-Asp₁₀.

49. The antibody of any of claims 44-47, wherein the bone-homing peptide is L-Asp₆.

50. The antibody of any of claims 44-49, wherein the one or more polypeptides comprise an adrenergic agonist, an anti-apoptosis factor, an apoptosis inhibitor, a cytokine receptor, a cytokine, a cytotoxin, an erythropoietic agent, a glutamic acid decarboxylase, a glycoprotein, a growth factor, a growth factor receptor, a hormone, a hormone receptor, an interferon, an interleukin, an interleukin receptor, a kinase, a kinase inhibitor, a nerve growth factor, a netrin, a neuroactive peptide, a neuroactive peptide receptor, a neurogenic factor, a neurogenic factor receptor, a neuropilin, a neurotrophic factor, a neurotrophin, a neurotrophin receptor, an N-methyl-D-aspartate antagonist, a plexin, a protease, a protease inhibitor, a protein decarboxylase, a protein kinase, a protein kinase inhibitor, a proteolytic protein, a proteolytic protein inhibitor, a semaphorin, a semaphorin receptor, a serotonin transport protein, a serotonin uptake inhibitor, a serotonin receptor, a serpin, a serpin receptor, or a tumor suppressor.

51. The antibody of any of claims 44-50, wherein the one or more polypeptides comprise a cytokine.

52. The antibody of claim 51, wherein the cytokine is IL-6.

53. The antibody of any of claims 44-52, wherein the one or more polypeptides are conjugated to a drug.

54. The antibody of claim 53, wherein the drug is monomethyl auristatin E (MMAE), ozogamicin, tiuxetan, vedotin, pasudotoxtdfx, vedotin, mafodotin, calicheamicin, maytansine, or deruxtecan.

55. The antibody of claim 53, wherein the drug is an anti-mitotic drug.

56. The antibody of claim 55, wherein the anti-mitotic drug is monomethyl auristatin E (MMAE).

57. The antibody of any of claims 44-22, wherein the bone-targeting composition has increased binding affinity to HA as compared to a polypeptide that does not comprise the bone-homing peptide.

58. The antibody of any of claims 44-57, wherein the bone-targeting composition has two-fold to three-fold higher binding affinity to HA as compared to a polypeptide that does not comprise the bone-homing peptide.

59. A method of treating or preventing bone disease in a subject comprising administering to the subject an effective amount of a bone-targeting composition of any of claims 44-57.

60. The method of claim 59, wherein the subject has bone cancer or bone metastasis.

61. The method of claim 60, wherein the bone cancer is Ewing sarcoma, osteosarcoma, or chondrosarcoma.

62. The method of claim 60, wherein the bone metastasis is from breast cancer, myeloma, renal cancer, lung cancer, prostate cancer, thyroid cancer, or bladder cancer.

63. The method of any of claims 59-62, wherein the breast cancer is triple-negative breast cancer.

64. The method of any of claims 62-63, wherein the breast cancer is HER2-negative breast cancer.

65. The method of any of claims 62-64, wherein the breast cancer is HER2-positive breast cancer.

65.1 The method of claim 60, wherein the bone disease is osteoporosis, osteomalacia, periodontitis, rheumatoid arthritis, metabolic bone disease, a parathyroid disorder, steroid-induced osteoporosis, chemotherapy-induced bone loss, pre-menopausal bone loss, fragility and recurrent fractures, renal osteodystrophy, bone infections, or Paget's disease.

66. The method of any of claims 59-68, wherein the bone-targeting composition results in increased concentration of therapeutic polypeptide at the bone tumor niche, inhibits cancer development in the bone, and/or limits secondary metastases to other organs.

67. The method of any of claims 59-66, wherein the bone-targeting composition results in decreased micrometastasis-induced osteolyic lesions.

68. The method of any of claims 59-67, wherein the method comprises further administering an additional anti-cancer therapy.

69. The method of claim 68, wherein the additional anti-cancer therapy comprises surgery, chemotherapy, radiation therapy, hormonal therapy, immunotherapy or cytokine therapy.

70. The method of claim 68, wherein the additional anti-cancer therapy comprises immunotherapy or chemotherapy.

71. Use of a bone-targeting composition of any of claims 44-57 for the treatment or prevention of bone tumors in a subject with cancer.

72. The use of claim 71, wherein the subject has bone cancer or bone metastasis.

73. The use of claim 72, wherein the bone cancer is Ewing sarcoma, osteosarcoma, or chondrosarcoma.

74. The use of claim 72, wherein the bone metastasis is from breast cancer, myeloma, renal cancer, lung cancer, prostate cancer, thyroid cancer, or bladder cancer.

75. A method for engineering a bone-targeting composition of any of claims 44-57 comprising inserting at least one bone-homing peptide at a permissive internal site of the polypeptide.

76. The method of claim 75, wherein the polypeptide comprises two, three, or four bone-homing peptides.

77. The method of claim 75 or 77, wherein the bone-homing peptide is L-Asp₆.
